(19) United States
(12) Patent Application Publication
(10) Pub. No.: US 2023/0302191 A1
(43) Pub. Date: Sep. 28, 2023

(54) COMPOSITIONS AND METHODS FOR SUSTAINED OXYGEN RELEASE TO ISCHEMIC TISSUES

(71) Applicant: Washington University, St. Louis, MO (US)

(72) Inventors: Hong Niu, St. Louis, MO (US); Ya Guan, St. Louis, MO (US); Jianjun Guan, St. Louis, MO (US)

(73) Assignee: Washington University, St. Louis, MO (US)

(21) Appl. No.: 18/162,387

(22) Filed: Jan. 31, 2023

Related U.S. Application Data
(60) Provisional application No. 63/304,800, filed on Jan. 31, 2022.

(51) Int. Cl. A61L 26/00; C08L 39/06; C08L 33/26

(52) U.S. Cl. CPC A61L 26/0004; A61L 26/0047; A61L 26/0014; C08L 39/06; A61L 26/008; C08L 33/26; A61L 2300/10; A61L 2300/254

(57) ABSTRACT

In one aspect, a composition for sustained release of oxygen to a tissue is disclosed that includes at least one core-shell oxygen release microsphere (ORM), wherein the core includes a water-soluble polymer-reactive oxygen species (ROS) complex and the shell includes a biodegradable polymer conjugated to a ROS-scavenging enzyme. In some aspects, the reactive oxygen species includes hydrogen peroxide (H₂O₂), the ROS-scavenging enzyme includes catalase, the water-soluble polymer includes polyvinylpyrrolidone (PVP), and the biodegradable polymer includes poly(N-isopropylacrylamide-co-2-hydroxyethyl methacrylate-co-acrylate-oligolactide-co-Nacryloxysuccinimide)[poly(NIPAAm-co-HEMA-co-AOLA-co-NAS)].

### CROSS-REFERENCE TO RELATED APPLICATIONS

This application claims the benefit of priority to U.S. Provisional Application No. 63/304,800, the content of which is incorporated by reference herein in its entirety.

### STATEMENT REGARDING FEDERALLY SPONSORED RESEARCH OR DEVELOPMENT

This invention was made with government support under AG056919, EB022018, HL138353, and HL138175 awarded by the National Institutes of Health. The government has certain rights in the invention.

### MATERIAL INCORPORATED-BY-REFERENCE

The Sequence Listing, “019931-US-NP_SEQ_LIST_SUBSTITUTE.xml” (file size=9.68 KB, generated 14 Jun. 2023), which is a part of the present disclosure, includes a computer-readable form comprising nucleotide and/or amino acid sequences of the present invention. The subject matter of the Sequence Listing is incorporated herein by reference in its entirety.

### FIELD OF THE INVENTION

The present disclosure generally relates to compositions and methods for the sustained delivery of oxygen to ischemic tissues.

### BACKGROUND OF THE INVENTION

Cutaneous diabetic wound healing is a complex process with three overlapping phases: inflammation, proliferation, and remodeling. Immediately after the precipitating injury, impaired vasculature impedes oxygen delivery to the wound, creating a hypoxic environment around the wound. This hypoxia is exacerbated by the recruitment of inflammatory cells with high oxygen consumption. Although acute hypoxia promotes cell proliferation and initiates tissue repair, long-term oxygen deprivation in chronic wounds impairs the healing process via inhibition of angiogenesis, reepithelialization, and extracellular matrix (ECM) synthesis. Thus, enhanced wound tissue oxygenation is key to chronic wound healing.

Diabetic wound healing has been clinically facilitated by oxygenation using existing oxygenation systems, such as hyperbaric oxygen therapy (HBOT). HBOT delivers 100% oxygen at 2 to 3 atm for 1 to 2 hours per treatment to patients with DFU. In some cases, HBOT promoted diabetic wound healing after 40 or more treatments, while in other studies, it did not show beneficial effects. Overall, the therapeutic efficacy of HBOT is widely considered to be inconsistent and unsatisfactory. This poor performance mainly results from HBOT's inability to continuously provide sufficient oxygen to the wounds because the oxygen content in the poorly vascularized wounds decreases quickly following the treatment. Moreover, as a systemic oxygen delivery strategy, HBOT may create risks of tissue hyperoxia, such as oxygen toxicity seizure.

To address the limitations of systemic oxygenation using HBOT, other existing oxygen-generating systems are configured to be implanted locally and increase the oxygen concentration in wound beds. These oxygen-generating systems were based on H₂O₂ (hydrogen peroxide), calcium peroxide, and perfluorocarbon. Localized oxygenation using such implants can avoid systemic hyperoxia while accelerating chronic wound healing. However, these oxygenation systems typically release oxygen for only 3 to 6 days, which is not long enough to facilitate the completion of diabetic wound healing. In addition, existing implant systems cannot release oxygen at the high rates needed to alleviate tissue hypoxia. Supplemental oxygen is required for sustained periods to facilitate important events associated with tissue healing, including angiogenesis, granulation, reepithelialization, and ECM synthesis, which can often take 2 weeks or longer. Hence, oxygen-generating systems are needed that continuously oxygenate the wound bed at high rates for long periods to accelerate healing.

### SUMMARY OF THE INVENTION

Among the various aspects of the present disclosure is the provision of compositions and methods for sustained oxygen release to ischemic tissues.

In one aspect, a composition for sustained release of oxygen to a tissue is disclosed that includes at least one core-shell oxygen release microsphere (ORM), wherein the core includes a water-soluble polymer-reactive oxygen species (ROS) complex and the shell includes a biodegradable polymer conjugated to a ROS-scavenging enzyme. In some aspects, the reactive oxygen species includes hydrogen peroxide (H₂O₂). In some aspects, the ROS-scavenging enzyme includes catalase. In some aspects, the water-soluble polymer includes polyvinylpyrrolidone (PVP). In some aspects, the biodegradable polymer includes poly(N-isopropylacrylamide-co-2-hydroxyethyl methacrylate-co-acrylate-oligolactide-co-Nacryloxysuccinimide)[poly(NIPAAm-co-HEMA-co-AOLA-co-NAS)]. In some aspects, the composition further includes a ROS-scavenging hydrogel. In some aspects, the ROS-scavenging hydrogel includes copolymerized NIPAAm, HEMA, and 4-(acryloyloxymethyl)-phenylboronic acid pinacol ester. In some aspects, the hydrogel includes at least one of a thermosensitive hydrogel, an injectable hydrogel, and any combination thereof. In some aspects, the tissue includes an ischemic tissue. In some aspects, the ischemic tissue includes a tissue associated with an ischemic condition selected from diabetes, peripheral artery disease, and coronary heart disease. In some aspects, the ischemic tissue includes a chronic diabetic wound bed.

In another aspect, a method for the sustained delivery of oxygen to a tissue is disclosed that includes administering the composition described above to the tissue. In some aspects, the tissue includes an ischemic tissue. In some aspects, the ischemic tissue includes a tissue associated with an ischemic condition selected from diabetes, peripheral artery disease, and coronary heart disease. In some aspects, the ischemic tissue includes a chronic diabetic wound bed.

In yet another aspect, a kit that includes at least one core-shell oxygen release microsphere (ORM) is disclosed. In one aspect the core can be a water-soluble polymer-reactive oxygen species (ROS) complex and the shell can be a biodegradable polymer conjugated to a ROS-scavenging enzyme. In another aspect, the biodegradable polymer in the kit can be poly(N-isopropylacrylamide-co-2-hydroxyethyl methacrylate-co-acrylate-oligolactide-co-Nacryloxysuccinimide)[poly(NIPAAm-co-HEMA-co-AOLA-co-NAS)]. In another aspect, the kit can include a ROS-scavenging hydrogel. In yet another aspect, the ROS-scavenging hydrogel in the kit can include copolymerized NIPAAm, HEMA, and 4-(acryloyloxymethyl)-phenylboronic acid pinacol ester. In another aspect, the kit can be used to treat a diabetic wound.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A is a schematic illustration of an ORM and its oxygen-release mechanism.

FIG. 1B is a scanning electron microscopy image of an ORM.

FIG. 1C is a fluorescent image of an ORM.

FIG. 1D is a fluorescent image of an ORM after catalase (labeled with FITC) conjugation. The color of FITC was adjusted.

FIG. 1E is a graph showing the oxygen-release kinetics of ORMs. n=8.

FIG. 1F is a graph showing the double-stranded DNA (dsDNA) content of HaCaT cells (n=5) cultured under hypoxia with and without ORMs.

FIG. 1G is a graph showing the double-stranded DNA (dsDNA) content of human dermal fibroblasts (HDFs; n=5) cultured under hypoxia with and without ORMs.

FIG. 1H is a graph showing the double-stranded DNA (dsDNA) content of human arterial endothelial cells (HAECs; n=3) cultured under hypoxia with and without ORMs.

FIG. 1I is a graph showing ROS content in HaCaT cells (n=10) cultured under normoxia, hypoxia, or hypoxia with ORM.

FIG. 1J is a graph showing ROS content in HDFs (n=6) (J) cultured under normoxia, hypoxia, or hypoxia with ORM.

FIG. 1K is a graph showing ROS content in HAECs (n=8) cultured under normoxia, hypoxia, or hypoxia with ORM.

FIG. 1L is a set of optical images showing the migration of HaCaT cells cultured under hypoxia with and without ORM. n=4.

FIG. 1M is a graph quantifying the migration of HaCaT cells cultured under hypoxia with and without ORM (n=4).

FIG. 1N is a graph of gene expression of PDGFB, VEGFA, and FGF2 in HaCaT cells cultured under hypoxia with and without ORM (n≥3).

FIG. 1O is a set of optical images showing the migration of HDFs cultured under hypoxia with and without ORM (n=4).

FIG. 1P is a graph quantifying migration of HDFs cultured under hypoxia with and without ORM (n=4).

FIG. 1Q is a graph of gene expression of PDGFB, VEGFA, and FGF2 in HDFs cultured under hypoxia with and without ORM (n≥3).

FIG. 1R is a schematic illustration of a HAEC tube formation assay.

FIG. 1S is a set of fluorescent images showing (S and T) Tube formation (S) and tube density (T) of HAECs cultured under hypoxia for 16 hours with and without ORM. Scale bars, 50 μm. DAPI, 4′,6-diamidino-2-phenylindole.

FIG. 1T is a graph of tube density (T) of HAECs cultured under hypoxia for 16 hours with and without ORM. *P<0.05, **P<0.01, and ***P<0.001. NS, not significant.

FIG. 2A is a schematic of the experimental setup of the EPR experiments to study the effect of ORMs on intracellular oxygen content in hypoxia-induced cells. Keratinocytes (HaCaT cells) were incubated with lithium phthalocyanine (LiPc) nanoparticles for endocytosis and then cultured under 1% oxygen conditions (with or without ORM) for 24 hours.

FIG. 2B shows representative EPR signals from experiments on cells with and without ORMs.

FIG. 2C is a graph of intracellular oxygen content in keratinocytes (HaCaT cells) as measured with EPR experiments. Keratinocytes (HaCaT cells) were incubated with lithium phthalocyanine (LiPc) nanoparticles for endocytosis and then cultured under 1% oxygen conditions (with or without ORM) for 24 hours. n=3 for each group. *P<0.05.

FIG. 2D is a graph of intracellular ATP content in HaCaT cells measured by an ATP assay kit. Cells were cultured under 1% oxygen conditions (with or without ORM) for 24 hours. **P<0.01.

FIG. 2E is a set of immunoblots of HO-1 and phosphorylated Erk1/2 (p-Erk1/2) in dermal fibroblasts cultured under hypoxic conditions for 48 hours with and without ORM. t-Erk1/2 was used as the internal reference. Glyceraldehyde-3-phosphate dehydrogenase (GAPDH) served as a loading control.

FIG. 3A is a schematic of the ROS-scavenging (ROSS) hydrogel capable of scavenging ROS in chronic wounds or generated from released oxygen or PVP/H2O2.

FIG. 3B is a schematic of the synthesis and ROS-scavenging mechanism of ROSS hydrogel. BPO denotes benzoyl peroxide.

FIG. 3C is a set of photos showing the injectability and gelation of ROSS hydrogel (Gel) and Gel/ORM construct.

FIG. 3D is a graph showing the scavenging effect on hydroxyl radicals of ROSS gel and non-ROS-responsive gel (Control); (n=4).

FIG. 3E is a graph showing the scavenging effect on superoxide of ROSS gel and non-ROS-responsive gel (Control); (n=3).

FIG. 3F is a graph showing degradation of ROSS gel at 37° C. for 4 weeks in DPBS with 0 and 50 mM H₂O₂.

FIG. 3G is a schematic illustration of an in vitro model for skin cell survival on gels under 100 μM H₂O₂ to mimic the in vivo cell environment under oxidative stress. Non-ROS-responsive gel was used as a control.

FIG. 3H is a graph of cell viability of HaCaT cells at 48 and 72 hours on control gel and ROSS gel (normalized to the initial cell viability on each gel). n≥6. *P<0.05 and ***P<0.001.

FIG. 4A is a schematic illustration of the design of animal experiments to test the therapeutic effect of ROSS gel (Gel) and ORMs in a db/db mouse model.

FIG. 4B is a set of representative images of the wounds treated with or without Gel and ORMs for 16 days.

FIG. 4C is a graph of wound size change at 16 days post-wounding with no treatment, gel, or gel/ORM applied. Wound size at each time point was normalized to day 0. n≥8. *P<0.05, **P<0.01, and ***P<0.001.

FIG. 4D is a set of immunofluorescence staining images of cytokeratin 10 (K10, green) and cytokeratin 14 (K14, red) for the wounds at days 8 and 16 with no treatment, gel, or gel/ORM applied. Scale bars, 200 μm.

FIG. 4E is a set of immunofluorescence staining images of K14 (red) in the wounded region at days 8 and 16 with no treatment, gel, or gel/ORM applied. Scale bars, 50 μm.

FIG. 4F is a graph showing the quantification of hair follicle density in the wounded region at days 8 and 16 with no treatment, gel, or gel/ORM applied. *P<0.05, **P<0.01, and ***P<0.001.

FIG. 4G is a set of hematoxylin and eosin staining images of the wounded skin at days 8 and 16 with no treatment, gel, or gel/ORM applied. Scale bars, 500 μm.

FIG. 4H is a graph showing the quantification of epidermal thickness. Epidermal thickness was calculated in the region where the wound was closed with no treatment, gel, or gel/ORM applied. *P<0.05, **P<0.01, and ***P<0.001.

FIG. 4I is a set of images of picrosirius red staining of the wounded skin at day 16 with no treatment, gel, or gel/ORM applied. Scale bar, 50 μm.

FIG. 4J is a graph showing the quantification of total collagen deposition on day 16. *P<0.05, **P<0.01, and ***P<0.001.

FIG. 4K is a graph of the quantification of the collagen I/III ratio at day 16 with no treatment, gel, or gel/ORM applied. *P<0.05, **P<0.01, and ***P<0.001.

FIG. 5A is a set of immunofluorescence staining images of Ki67 (red) in the wounded region at 8 and 16 days after wounding with no treatment, gel, or gel/ORM applied. Scale bars, 50 μm.

FIG. 5B is a set of immunofluorescence staining images of PGC1α (green) in the wounded region at 8 and 16 days after wounding with no treatment, gel, or gel/ORM applied. Scale bars, 50 μm.

FIG. 5C is a graph of the quantification of Ki67+ cell density with no treatment, gel, or gel/ORM applied. *P<0.05, **P<0.01, and ***P<0.001.

FIG. 5D is a graph of the quantification of PGC1α+ cell density with no treatment, gel, or gel/ORM applied. *P<0.05, **P<0.01, and ***P<0.001.

FIG. 5E is a graph of gene expression of Pdgfb from the tissue lysates extracted from the wounded skin on day 8 with no treatment, gel, or gel/ORM applied. n≥4. *P<0.05, **P<0.01, and ***P<0.001.

FIG. 5F is a graph of gene expression of Vegfa (F) from the tissue lysates extracted from the wounded skin on day 8 with no treatment, gel, or gel/ORM applied. n≥4. *P<0.05, **P<0.01, and ***P<0.001.

FIG. 5G is a set of immunofluorescence staining images of isolectin (green) in the wounded region at 8 and 16 days after wounding with no treatment, gel, or gel/ORM applied. Scale bars, 50 μm. Nuclei were stained with DAPI in all immunofluorescence staining images.

FIG. 5H is a graph of quantification of vessel density of wounded sites with no treatment, gel, or gel/ORM applied. *P<0.05, **P<0.01, and ***P<0.001.

FIG. 6A is a set of immunofluorescence staining images of CM-H2DCFDA (red) at the wounded site on days 8 and 16 with no treatment, gel, or gel/ORM applied. Scale bars, 50 μm.

FIG. 6B is a set of immunofluorescence staining images of CD86 (red) at the wounded site on days 8 and 16 with no treatment, gel, or gel/ORM applied. Scale bars, 50 μm. Nuclei were stained with DAPI in all immunofluorescence staining images.

FIG. 6C is a graph of the quantification of ROS+ cell density with no treatment, gel, or gel/ORM applied. The results were normalized to the ROS+ cell density in the no-treatment group on day 8. n≥8. *P<0.05 and ***P<0.001.

FIG. 6D is a graph of the quantification of CD86+ cell density within wound sites with no treatment, gel, or gel/ORM applied. *P<0.05 and ***P<0.001.

FIG. 6E is a cytokine array analysis of the proinflammatory cytokine level in the wounds with gel or gel/ORM obtained 8 days after treatment.

FIG. 6F is a graph of the quantitative summary of cytokine array analysis summarized in FIG. 6E. A.U., arbitrary units.

FIG. 7 is a set of immunoblots of HO-1 and p-Erk1/2 from the tissue lysates extracted from wounded skin at days 8 and 16 after wounding with gel or gel/ORM applied. t-Erk1/2 was used as the internal reference. GAPDH was used as a loading control.

FIG. 8 is a schematic of the mechanisms of accelerated wound healing by ORMs encapsulated in ROS-scavenging hydrogel. The ORMs and ROSS gel augmented cell proliferation and migration, promoted angiogenesis and reepithelialization, and decreased inflammation and oxidative stress in diabetic wounds.

FIG. 9 is a ¹H-NMR graph of poly(NIPAAm-co-HEMA-co-AOLA-co-NAS) showing the characterization of the chemical structure of the shell polymer.

FIG. 10 is a set of fluorescent images showing the characterization of cellular ROS content in HaCaT cells. HaCaT cells were stained with CM-H2DCFDA (green) and DAPI after culturing under normoxia, hypoxia, or hypoxia with ORM for 3 days. Scale bar=50 μm.

FIG. 11 is a set of fluorescent images showing the characterization of cellular ROS content in HDFs. HDFs were stained with CM-H2DCFDA (green) and DAPI after culturing under normoxia, hypoxia, or hypoxia with ORM for 5 days. Scale bar=50 μm.

FIG. 12 is a set of fluorescent images showing the characterization of cellular ROS content in HAECs. HAECs were stained with CM-H2DCFDA (green) and DAPI after culturing under normoxia, hypoxia, or hypoxia with ORM for 3 days. Scale bar=50 μm.

FIG. 13 is a 1H-NMR of poly(NIPAAm-co-HEMA-co-4-(acryloxymethyl)-phenylboronic-acid-pinacol-ester showing the characterization of the chemical structure of ROSS gel.

FIG. 14 is a set of immunofluorescence images showing in vivo keratinocyte migration in diabetic wounds without treatment, or treated with Gel or Gel/ORM. Immunofluorescence staining of cytokeratin 10 (K10, green) and cytokeratin 14 (K14, red) of the wounds on day 8 and day 16. Scale bar=200 μm.

FIG. 15 summarizes the primer sequences used in real-time RT-PCR for the in vitro and in vivo studies disclosed herein.

### DETAILED DESCRIPTION OF THE INVENTION

Non-healing diabetic wounds are common complications for diabetic patients. Chronic hypoxia is a prominent characteristic of diabetic wounds and a severe impediment to the healing process. Consequently, sustained oxygenation of skin cells to mitigate chronic hypoxia represents an approach to accelerating diabetic wound healing. Oxygen therapy is advantageous over drug therapy because it raises fewer toxicity concerns. However, existing oxygen therapy approaches cannot provide sufficient oxygen to metabolic-demanding skin cells long enough to promote diabetic wound healing.

Because chronic hypoxia prominently delays wound healing, sustained oxygenation to alleviate hypoxia is hypothesized to promote diabetic wound healing. However, sustained oxygenation has not been achieved by current clinical approaches such as hyperbaric oxygen therapy. A sustained oxygenation composition that includes oxygen-release microspheres and a reactive oxygen species (ROS)-scavenging hydrogel is disclosed herein in various aspects. The hydrogel captures the naturally elevated ROS in diabetic wounds, which may be further elevated by the oxygen released from the administered microspheres. Without being limited to any particular theory, the sustained release of oxygen provided by the disclosed composition is thought to augment the survival and migration of keratinocytes and dermal fibroblasts, promote angiogenic growth factor expression and angiogenesis in diabetic wounds, and decrease proinflammatory cytokine expression. The effects described above combine to significantly increase the wound closure rate. The experimental results described in the Experiments herein demonstrate that sustained oxygenation alone, without using drugs, promotes the healing of diabetic wounds.

In various aspects, an oxygen-release composition that continuously oxygenates diabetic wounds is disclosed herein. The disclosed oxygen-release composition is configured to release molecular oxygen and scavenge reactive oxygen species (ROS) simultaneously. The disclosed composition includes oxygen-release microspheres (ORMs) and their carrier a reactive oxygen species-scavenging hydrogel (ROSS gel). The ROSS gel is injectable, thermosensitive, and fast-gelling to facilitate administering the composition by direct injection into the wound bed. The ORMs have a core-shell structure that includes a PVP/H₂O₂ complex as the core and a bioeliminable polymer as the shell. The high-molecular weight PVP/H₂O₂ complex reduces H₂O₂ diffusivity to provide for sustained release of H₂O₂ during the hydrolysis of the shell polymer. The ORM surface is conjugated with catalase to timely convert H₂O₂ in the released PVP/H₂O₂ into molecular oxygen. Consequently, the ORMs directly release oxygen, whereas existing oxygen-release systems typically release H₂O₂ instead of oxygen.

In various aspects, the microspheres are configured to release oxygen for at least 2 weeks or more. The injectable, thermosensitive, and fast-gelling ROSS hydrogels used as the ORM carrier largely retain the ORMs in the diabetic wounds after delivery. The ROS-scavenging hydrogel eliminates the H₂O₂ in the released PVP/H₂O₂, even if the H₂O₂ is not completely converted to oxygen by the catalase at the ORM surface. In addition, the ROS-scavenging hydrogel captures the H₂O₂ that is typically upregulated in diabetic wounds to decrease oxidative stress and accelerate wound healing.

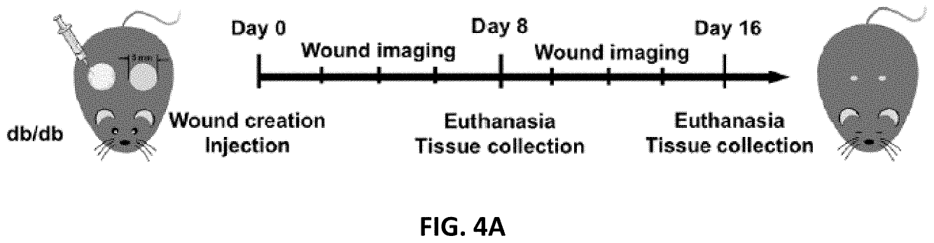

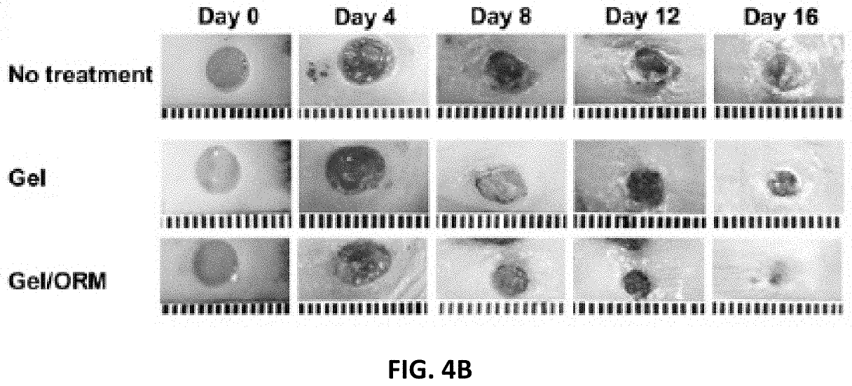

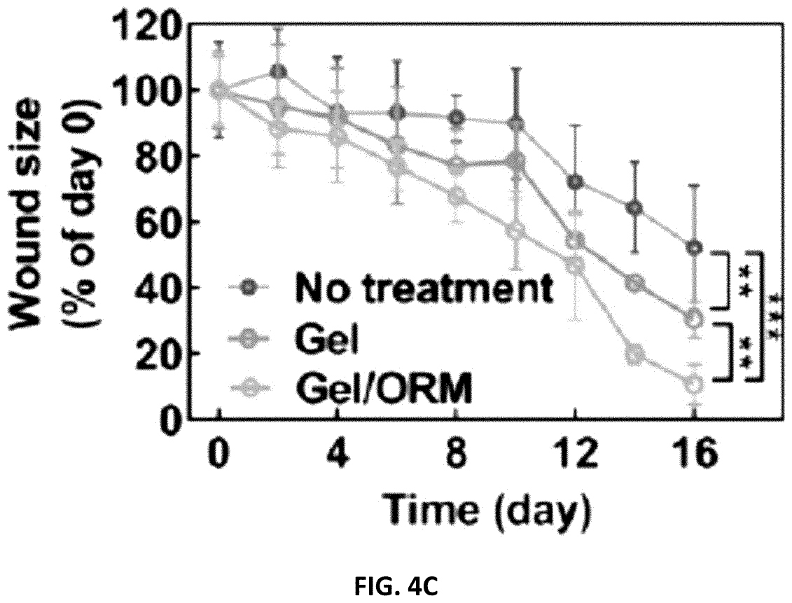

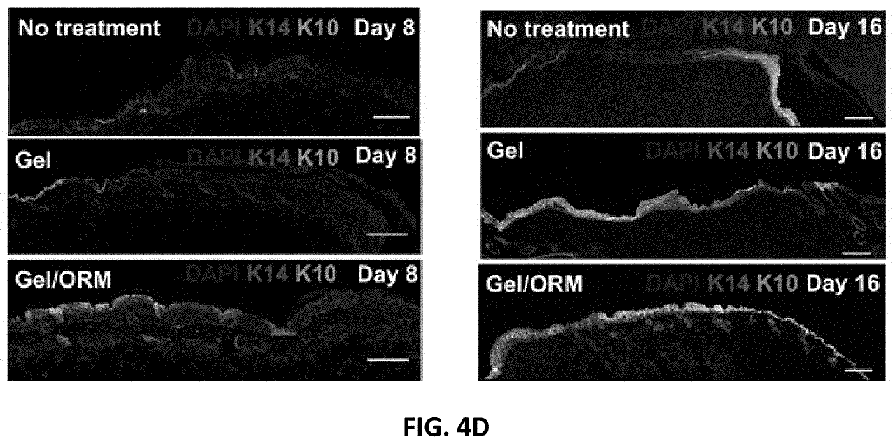

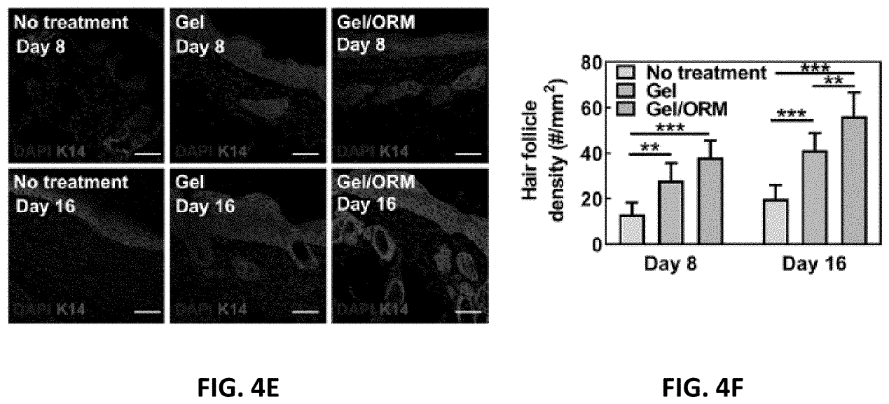

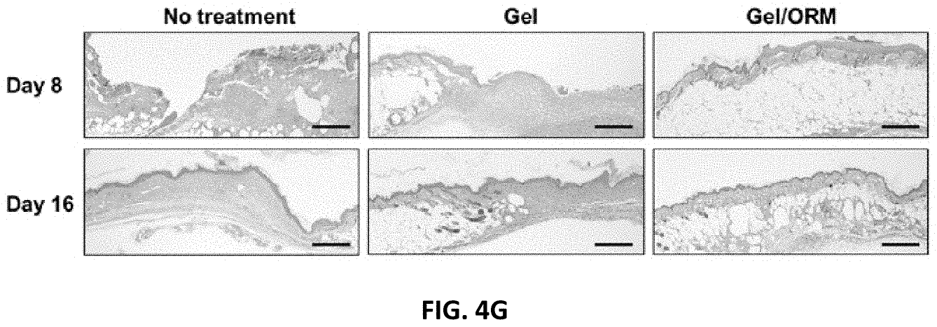

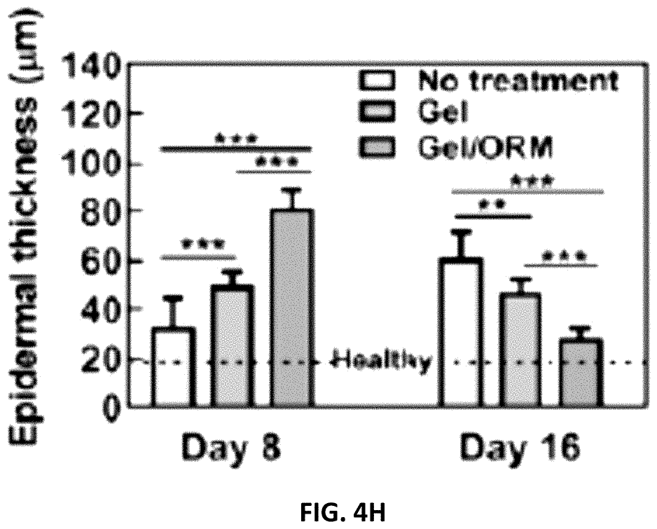

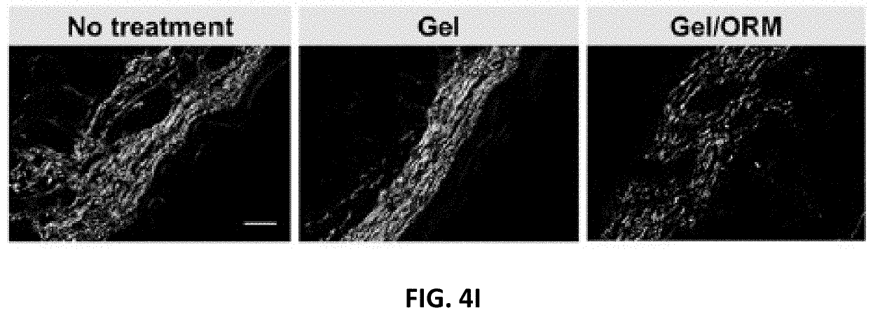

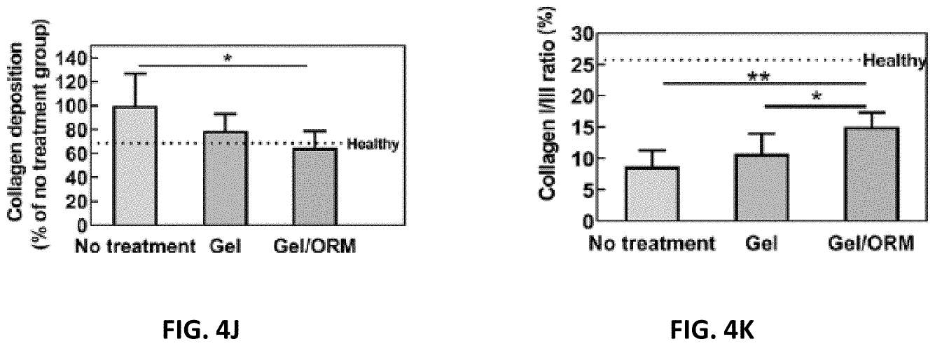

As described in the Examples herein, the efficacy and mechanism of action of the oxygen-release system in healing excisional wounds in db/db mice were evaluated (see FIG. 4A of Appendix A). This model exhibited a significant delay in wound closure and impaired wound bed vascularization compared with other well-accepted murine diabetes models, such as streptozocin-induced C57BL/6J and Akita mice. Administration of the disclosed oxygen-release composition to the wounds of the db/db mice substantially promoted wound closure (see FIG. 4C of Appendix A). The accelerated wound healing was caused by the sustained oxygenation and ROS scavenging in diabetic wounds that augmented cell survival, accelerated cell migration, stimulated angiogenesis, and reduced oxidative stress and inflammation, as demonstrated in the Examples herein.

The results of the experiments described in the Examples herein demonstrate that administration of the sustained oxygen-release composition as disclosed herein substantially accelerated diabetic wound closure. The simultaneous sustained release of oxygen to the wound tissues and scavenging of reactive oxidative species from the wound tissues provided by the composition as disclosed herein promoted skin cell survival, migration, and paracrine effects; stimulated endothelial tube formation and angiogenesis; and decreased tissue inflammation. Treatment using the disclosed oxygen-release composition resulted in wound closure rates that matched or exceed wound closure rates achieved in the same animal model using existing compositions that used growth factors or exogenous cells to treat impaired wound healing. Treatment using the disclosed sustained oxygen-release composition represents an effective therapeutic approach for accelerated healing of chronic diabetic wounds without using drugs.

Beyond wound healing, the developed oxygen-release system may be used to treat other ischemic diseases, such as peripheral artery disease and coronary heart disease.

ORMs

In various aspects, the disclosed sustained oxygen-release composition includes a plurality of oxygen-release microspheres (ORMs) configured to produce oxygen after the composition is implanted or injected into a wound bed within an ischemic tissue. In various aspects, each ORM is a microsphere comprising a core encapsulated within a shell.

In various aspects, the core comprises an oxygen-releasing species conjugated to a water-soluble polymer. The oxygen-releasing species may comprise any suitable oxygen-releasing compound without limitation including, but not limited to hydrogen peroxide, calcium peroxide, and perfluorocarbon. In one exemplary aspect, the oxygen-releasing compound is hydrogen peroxide. The water-soluble polymer comprises any suitable water-soluble polymer without limitation including, but not limited to, polyvinyl pyrrolidone (PVP).

In various aspects, the shell of each ORM comprises a bioeliminable and bioconjugatable polymer and further includes a catalytic compound conjugated to the polymer that is configured to catalyze the conversion of the oxygen-releasing species to oxygen as the oxygen-releasing species conjugated to a water-soluble polymer is released from the core through the shell. Any suitable bioeliminable and bioconjugatable polymer may be used for the shell of the ORM without limitation including, but not limited to, poly(N-isopropylacrylamide-co-2-hydroxyethyl methacrylate-co-acrylate-oligolactide-co-N-acryloxysuccinimide) [poly(NIPAAm-co-HEMA-co-AOLA-co-NAS)]. Any suitable catalytic compound conjugated to the polymer may be conjugated to the polymer of the shell without limitation including, but not limited to, catalase.

In various aspects, the ORMs have microsphere diameters ranging from about 1 μm to about 50 μm. In various other aspects, the microsphere diameters range from about 1 μm to about 3 μm, from about 2 μm to about 4 μm, from about 3 μm to about 5 μm, from about 4 μm to about 6 μm, from about 5 μm to about 15 μm, from about 10 μm to about 20 μm, from about 15 μm to about 25 μm, from about 20 μm to about 30 μm, from about 25 μm to about 35 μm, from about 30 μm to about 40 μm, from about 35 μm to about 45 μm, and from about 40 μm to about 50 μm. In one aspect, the ORMs have a microsphere diameter of about 5 μm.

In some aspects, the ORMs are configured to release oxygen for at least one week, at least 1.5 weeks, at least 2 weeks, at least 2.5 weeks, and at least 3 weeks. In one aspect, the ORMs are configured to release oxygen for about 2 weeks.

In some aspects, the ORMs of the disclosed composition are configured to release an amount of oxygen sufficient to maintain an oxygen level ranging from about 5% to about 20% within the wound bed, In other aspects, the maintained oxygen level is at least about 5%, at least about 6%, at least about 8%, at least about 10%, at least about 12%, at least about 15%, and at least about 20%. In one aspect, the ORMs of the disclosed composition are configured to release an amount of oxygen sufficient to maintain an oxygen level of about 10% within the wound bed.

In some aspects, the catalase conjugated to the polymer of the ORM coating is configured to maintain the concentration of the oxygen-releasing species released at concentrations that are below levels that may cause adverse events within the wound bed including, but not limited to, cell apoptosis. In one aspect, the catalase conjugated to the polymer of the ORM coating is configured to maintain the concentration of H₂O₂ to below about 10 mM.

In various aspects, the ORMS may be fabricated using any suitable method without limitation including, but not limited to, the double emulsion method described in the Examples below.

Hydrogel

In various aspects, the sustained oxygen release composition includes the ORMs described above suspended within an injectable and thermosensitive hydrogel with a fast gelation rate to facilitate implantation of the composition by directly injecting it into the wound bed. In addition to serving as a delivery vehicle for the ORMs, the injectable and thermosensitive hydrogel is further configured to scavenge reactive oxygen species (ROS) inadvertently released from the ORMs without being converted to oxygen by the catalase in the ORM coating, as well as scavenging upregulated ROS within the diabetic wounds to protect skin cells from ROS-induced apoptosis. Any suitable injectable and thermosensitive hydrogel with a fast gelation rate and ability to scavenge ROS may be used without limitation including, but not limited to, copolymerized NIPAAm, HEMA, and 4-(acryloyloxymethyl)-phenylboronic acid pinacol ester.

In various aspects, the hydrogel may contain the ORMs at any suitable concentration without limitation. In some aspects, the ORMS are included at a concentration ranging from about 10 mg of ORMs/mL of hydrogel to about 100 mg of ORMs/mL of the hydrogel. In other aspects, the ORMS are included at a concentration ranging from about 10 mg/mL to about 20 mg/mL from about 15 mg/mL to about 25 mg/mL from about 20 mg/mL to about 30 mg/mL from about 25 mg/mL to about 35 mg/mL from about 30 mg/mL to about 40 mg/mL from about 35 mg/mL to about 45 mg/mL from about 40 mg/mL to about 50 mg/mL from about 45 mg/mL to about 55 mg/mL from about 50 mg/mL to about 60 mg/mL from about 55 mg/mL to about 65 mg/mL from about 60 mg/mL to about 70 mg/mL from about 65 mg/mL to about 75 mg/mL from about 70 mg/mL to about 80 mg/mL from about 75 mg/mL to about 85 mg/mL from about 80 mg/mL to about 90 mg/mL from about 85 mg/mL to about 96 mg/mL and from about 90 mg/mL to about 100 mg/mL. In one aspect, the ORMS are included at a concentration of about 40 mg/m L.

In various aspects, the hydrogel may be formed using any suitable method including, but not limited to a free radical polymerization of NIPAAm, HEMA, and 4-(acryloxymethyl)-phenylboronic acid pinacol ester using BPO as initiator and 1,4-dioxane as a solvent, as described in the Examples herein.

Formulation

The agents and compositions described herein can be formulated in any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption-delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce the dosage frequency.

Controlled-release preparations can also be used to modulate the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of the agent being metabolized or excreted from the body. The controlled release of an agent may be stimulated by various inducers, e.g., a change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of treating, preventing, or reversing wounds within ischemic tissues including, but not limited to, chronic diabetic wounds in a subject in need of administration of a therapeutically effective amount of a sustained oxygen release composition, so as to simultaneously provide supplemental oxygen and scavenge reactive oxygen species (ROS) to/from the ischemic tissue.

Methods described herein are generally performed on a subject in need thereof. A subject in need of the therapeutic methods described herein can be a subject having, diagnosed with, suspected of having, or at risk for developing wounds within ischemic tissues, including, but not limited to, a subject diagnosed with a diabetic condition. A determination of the need for treatment will typically be assessed by a history, physical exam, or diagnostic test consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of the disclosed sustained oxygen release composition is, for example, an amount that would cause the desired therapeutic effect in a subject while minimizing undesired side effects. In various embodiments, an effective amount of the disclosed sustained oxygen release composition described herein can substantially promote wound healing, slow the progress of wound growth, or limit the development or extent of a wound bed within ischemic tissue.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of the disclosed sustained oxygen release composition can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to promote wound healing within ischemic tissue.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single-dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from the compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of the disclosed sustained oxygen release composition can occur as a single event or over a time course of treatment. For example, the disclosed sustained oxygen release composition can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accordance with the methods described herein can be performed prior to, concurrent with, or after conventional treatment modalities for any other condition related to or unrelated to the ischemic condition associated with the wound to be treated including, but not limited to, a diabetic condition, peripheral artery disease, and coronary heart disease.

A sustained oxygen release composition can be administered simultaneously or sequentially with another agent, such as an antibiotic, an anti-inflammatory, or another agent. For example, the disclosed sustained oxygen release composition can be administered simultaneously with another agent, such as an antibiotic or an anti-inflammatory. Simultaneous administration can occur through the administration of separate compositions, each containing one or more of the disclosed sustained oxygen release composition, an antibiotic, an anti-inflammatory, or another agent. Simultaneous administration can occur through the administration of one composition containing two or more of the disclosed sustained oxygen release composition, an antibiotic, an anti-inflammatory, or another agent. The disclosed sustained oxygen release composition can be administered sequentially with an antibiotic, an anti-inflammatory, or another agent. For example, the disclosed sustained oxygen release composition can be administered before or after the administration of an antibiotic, an anti-inflammatory, or another agent.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. In some aspects, the disclosed sustained oxygen release composition is administered by implantation within a wound bed in an ischemic tissue. In other aspects, the disclosed sustained oxygen release composition is administered by direct injection into the wound bed in an ischemic tissue.

Agents and compositions described herein can be administered in a variety of methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve the taste of the product; or improve the shelf life of the product.

Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to the core-shell oxygen release microspheres (ORMs), the ROS-scavenging hydrogel, and any combination thereof as disclosed herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Sustained Oxygenation Accelerates Diabetic Wound Healing by Promoting Epithelialization and Angiogenesis and Decreasing Inflammation

To demonstrate the oxygen-release abilities and wound-healing efficacy of the sustained oxygen-release compositions described herein, the following experiments were conducted.

### Abstract:

Nonhealing diabetic wounds are common complications for diabetic patients. Because chronic hypoxia prominently delays wound healing, sustained oxygenation to alleviate hypoxia is hypothesized to promote diabetic wound healing. However, sustained oxygenation cannot be achieved by current clinical approaches, including hyperbaric oxygen therapy. Here, a sustained oxygenation system consisting of oxygen-release microspheres and a reactive oxygen species (ROS)-scavenging hydrogel is presented. The hydrogel captures the naturally elevated ROS in diabetic wounds, which may be further elevated by the oxygen released from the administered microspheres. The sustained release of oxygen augmented the survival and migration of keratinocytes and dermal fibroblasts, promoted angiogenic growth factor expression and angiogenesis in diabetic wounds, and decreased the proinflammatory cytokine expression. These effects significantly increased the wound closure rate. The findings demonstrate that sustained oxygenation alone, without using drugs, can heal diabetic wounds.

### Introduction:

Diabetes is a chronic metabolic disorder affecting 34.2 million people across the United States. One of the common complications of diabetes is diabetic foot ulcers (DFUs). Roughly 25% of diabetic patients experience DFU, which causes prolonged pain, decreased vitality, and possibly the need for foot amputation. Conservative medical care for DFU, such as wound off-loading, wound debridement, and infection control, has inconsistent outcomes and is associated with undesired side effects. Stable and safe treatments for chronic, nonhealing diabetic wounds could benefit millions of people.

Cutaneous diabetic wound healing is a complex process with three overlapping phases: inflammation, proliferation, and remodeling. Immediately after the precipitating injury, impaired vasculature impedes oxygen delivery to the wound, creating a hypoxic environment around the wound. This hypoxia is exacerbated by the recruitment of inflammatory cells with high oxygen consumption. Although acute hypoxia promotes cell proliferation and initiates tissue repair, long-term oxygen deprivation in chronic wounds impairs the healing process via inhibition of angiogenesis, reepithelialization, and extracellular matrix (ECM) synthesis. Thus, enhanced wound tissue oxygenation is key to chronic wound healing.

In the past few decades, diabetic wound healing has been clinically facilitated by oxygenation, particularly hyperbaric oxygen therapy (HBOT). HBOT delivers 100% oxygen at 2 to 3 atm for 1 to 2 hours per treatment to patients with DFU. In some cases, HBOT promoted diabetic wound healing after 40 or more treatments, while in other studies, it did not show beneficial effects. Overall, the therapeutic efficacy of HBOT is widely considered to be inconsistent and unsatisfactory. This poor performance mainly results from HBOT's inability to continuously provide sufficient oxygen to the wounds because the oxygen content in the poorly vascularized wounds decreases quickly following the treatment. Moreover, as a systemic oxygen delivery strategy, HBOT may create risks of tissue hyperoxia, such as oxygen toxicity seizure.

Several previous reports have developed oxygen-generating systems that are implanted locally to increase the oxygen concentration in wound beds to address the limitations of systemic oxygenation. These oxygen-generating systems were based on H2O2 (hydrogen peroxide), calcium peroxide, and perfluorocarbon. The localized oxygenation can avoid systemic hyperoxia while accelerating chronic wound healing. However, these oxygenation systems typically release oxygen for only 3 to 6 days, which is not long enough for diabetic wound healing. In addition, these systems cannot quickly release sufficient oxygen to relieve hypoxia. Oxygen is required for important events, including angiogenesis, granulation, reepithelialization, and ECM synthesis, which often take 2 weeks or longer. Hence, it is critical to develop oxygen-generating systems that continuously oxygenate the wound bed for long periods to accelerate healing.

Here, an oxygen-generating system based on oxygen-release microspheres (ORMs) and an injectable, fast-gelling, and reactive oxygen species (ROS)-scavenging hydrogel (ROSS gel) is presented. The microspheres quickly release enough oxygen to support cell survival under hypoxia and sustain the release of oxygen for at least 2 weeks. Unlike most current oxygen-generating systems that first release toxic H₂O₂ into the tissue environment and then rely on its decomposition to release oxygen, the designed ORMs directly release oxygen. The injectable and fast-gelling hydrogel is used as a carrier to deliver the microspheres into wounds and quickly immobilize them there, with a high tissue retention rate. The hydrogel has a relatively high water content, enabling it to maintain a moist environment surrounding the wound. Because it can also scavenge ROS, the oxygen-generating system can capture excessive ROS in diabetic wounds. While ROS plays a crucial role in regulating biological and physiological processes, elevated ROS in diabetic wounds can damage cells. In this study, we assessed the therapeutic efficacy of the oxygen-generating system in promoting wound closure was assessed. The underlying mechanisms of how sustained oxygen release promotes diabetic wound healing were also elucidated. While previous studies have shown that short-term oxygen release facilitated wound closure, the underlying mechanisms were not fully clear and were mainly attributed to wound angiogenesis. The effect of sustained oxygen release on skin cell survival, migration, and paracrine effects; intracellular oxygen content; prosurvival pathways; tissue angiogenesis; and tissue inflammation and oxidative stress were comprehensively evaluated.

### Results:

### Microspheres Released Molecular Oxygen Continuously

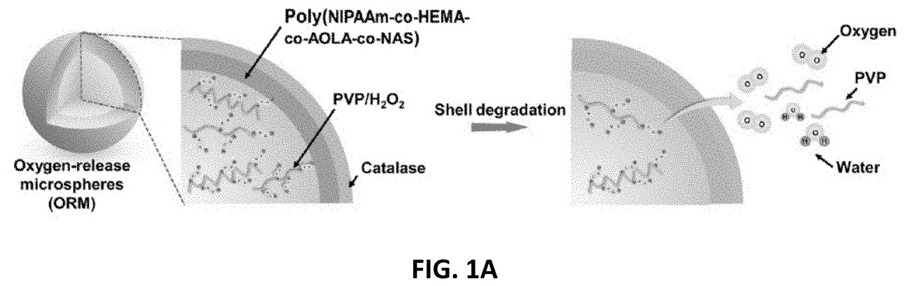

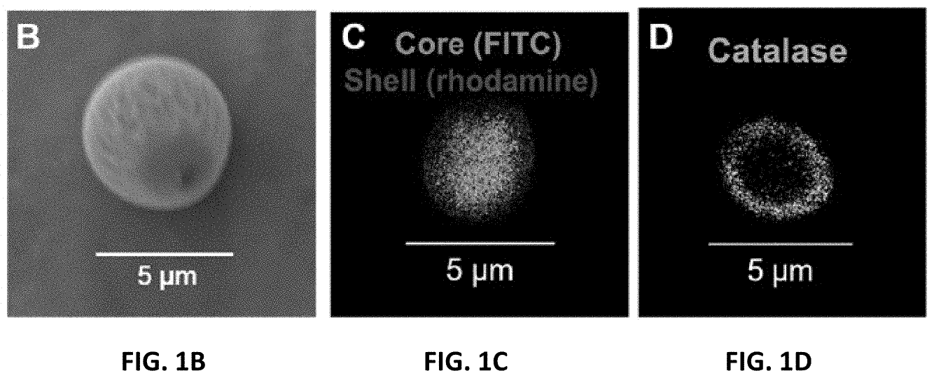

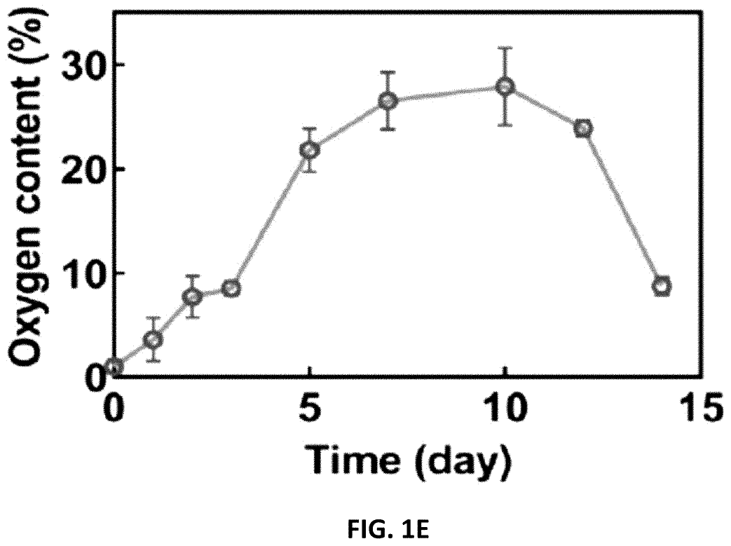

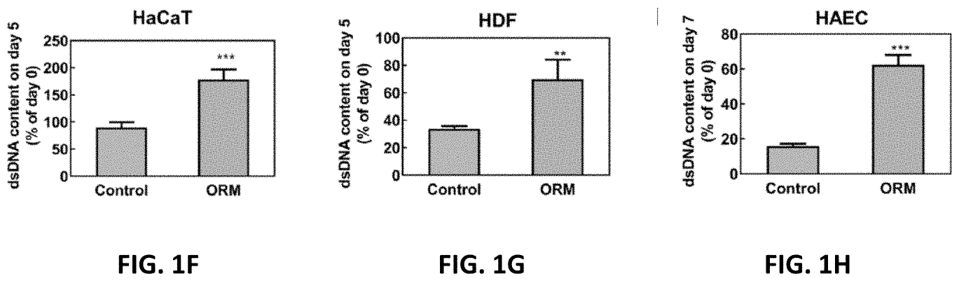

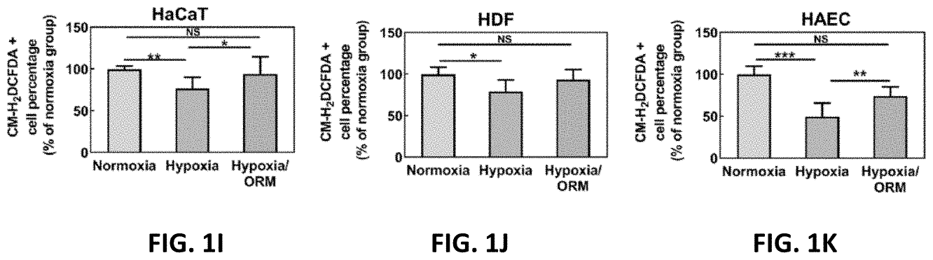

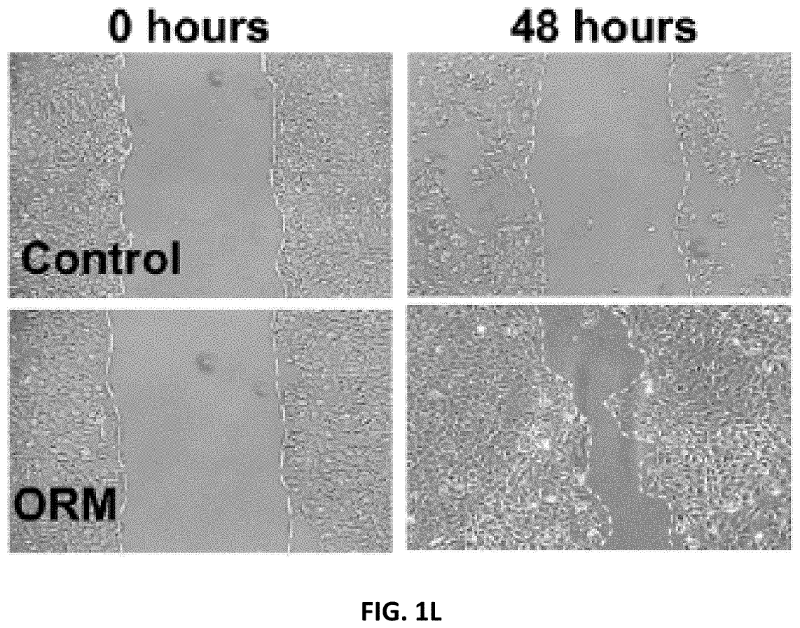

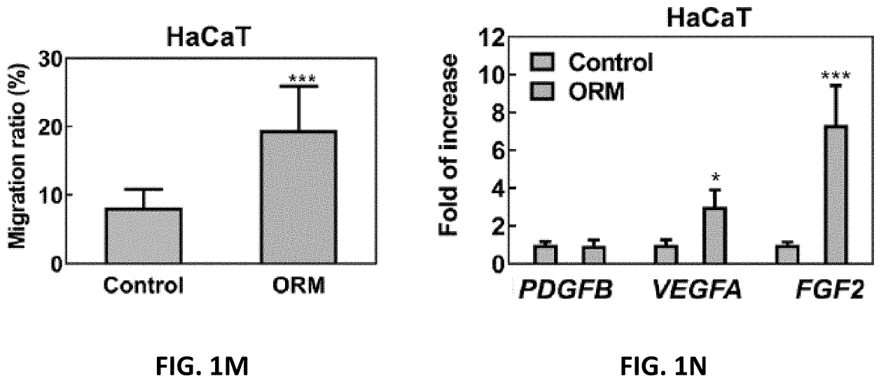

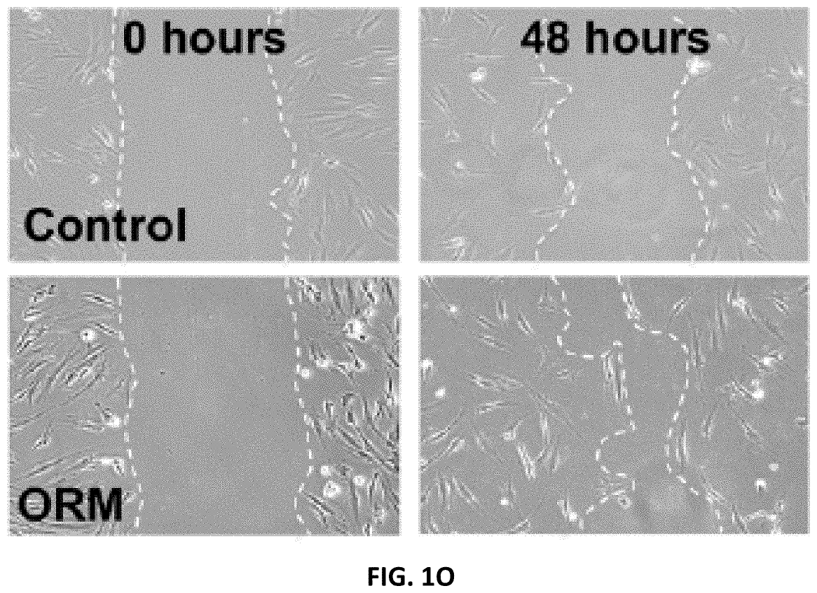

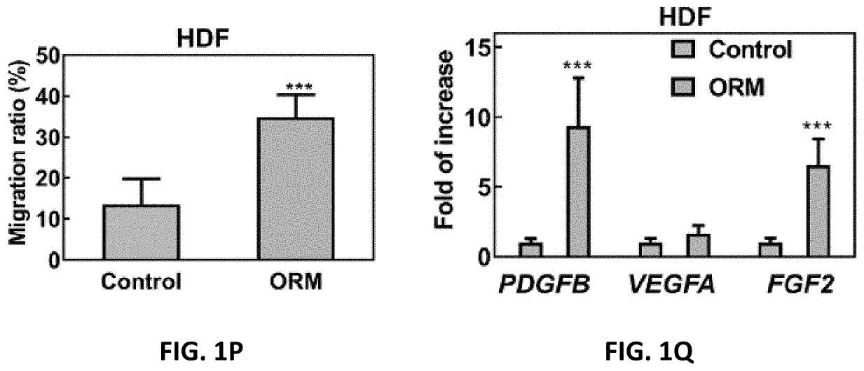

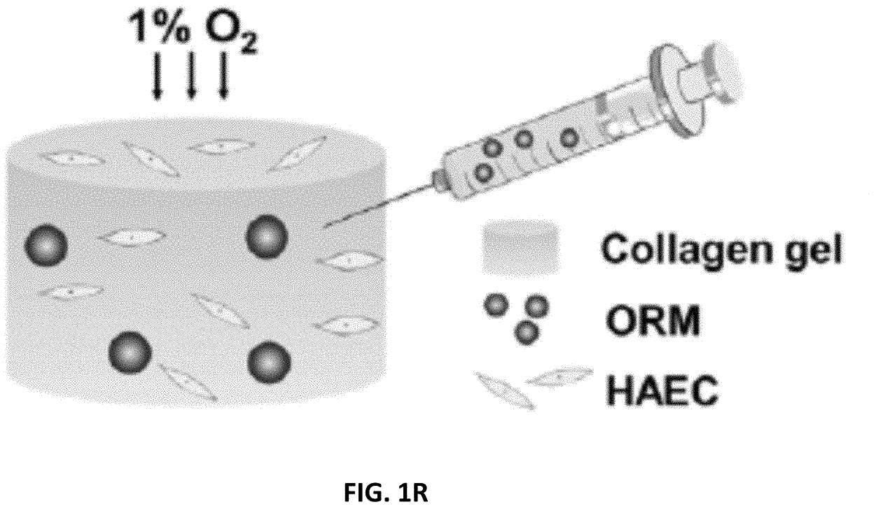

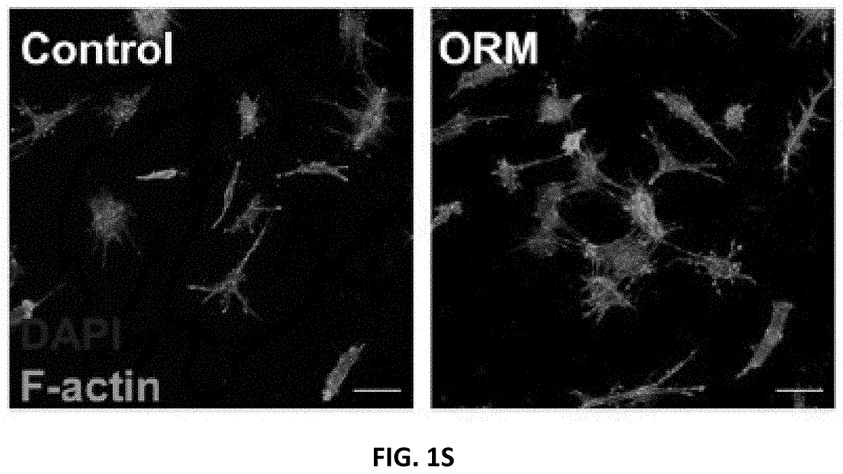

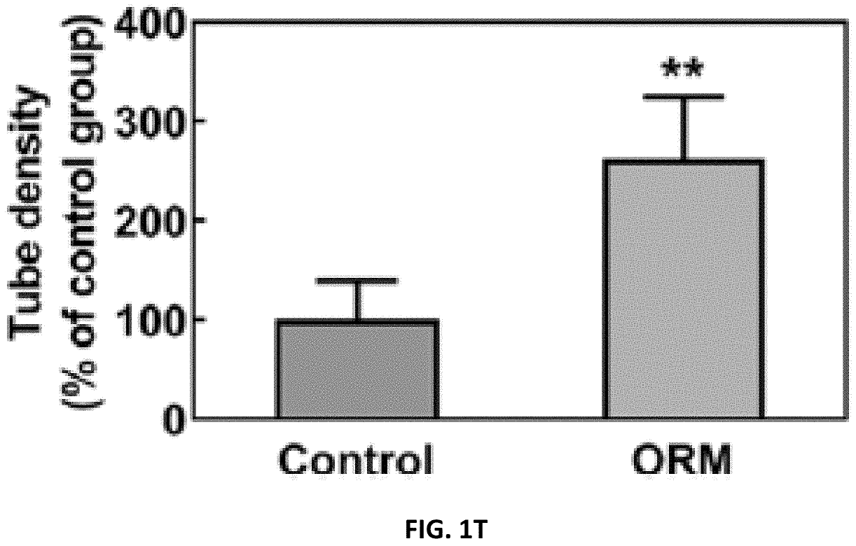

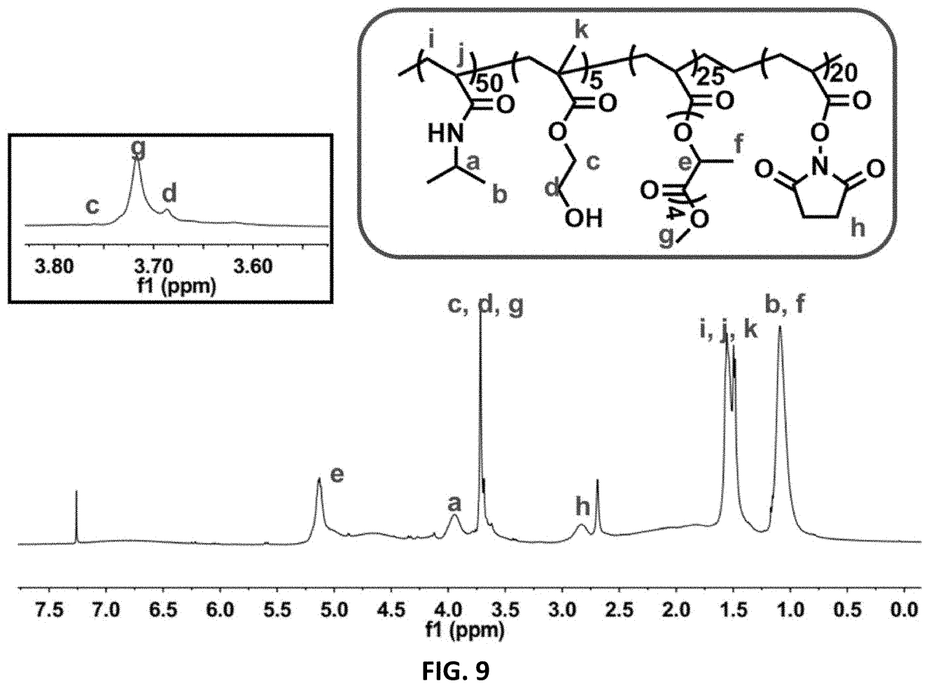

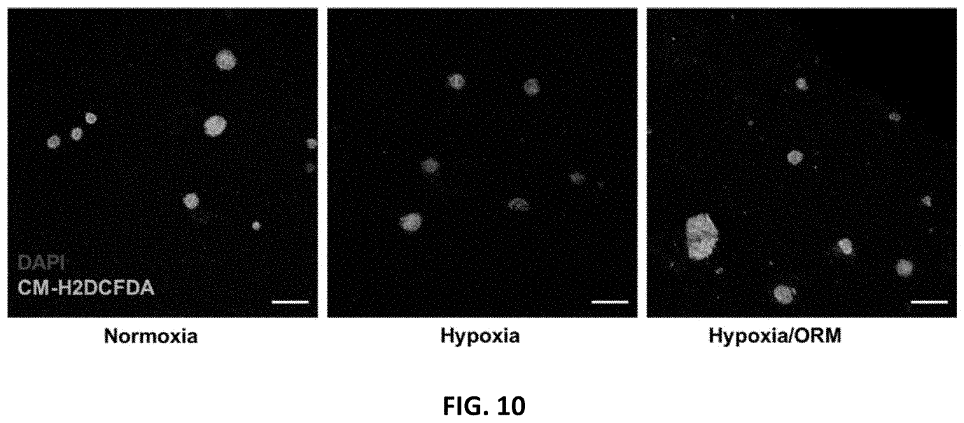

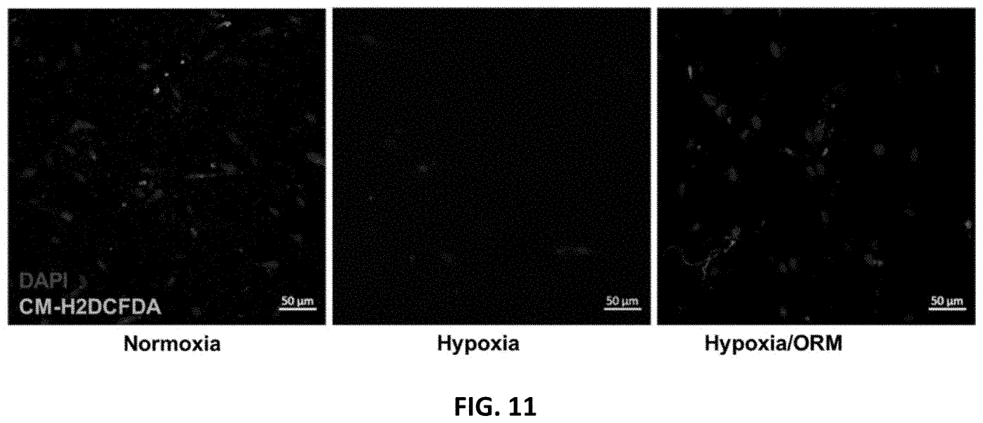

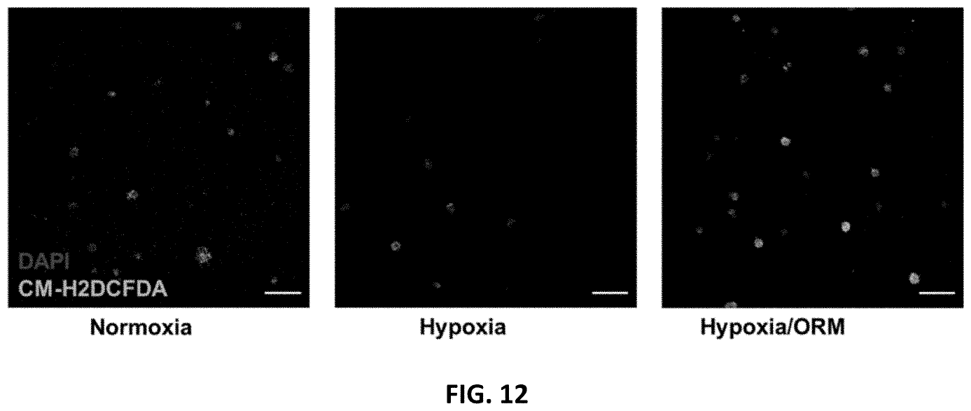

The ORMs had a core-shell structure (FIG. 1A), in which the core was a stable polyvinylpyrrolidone (PVP)/H₂O₂ complex. The shell was bioeliminable and bioconjugatable poly(N-isopropylacrylamide-co-2-hydroxyethyl methacrylate-co-acrylate-oligolactide-co-N-acryloxysuccinimide) [poly(NIPAAm-co-HEMA-co-AOLA-co-NAS)] (FIG. 9). The microspheres had a diameter of ˜5 μm (FIG. 1B). The core-shell structure was confirmed by fluorescent images (FIG. 1C). The shell of the microspheres was conjugated with catalase to timely convert the H₂O₂ in the released PVP/H₂O₂ into molecular oxygen and keep H₂O₂ from exiting the microspheres and causing toxicity concerns. A layer of catalase was conjugated on the shell, as evidenced by the fluorescence signal of fluorescein isothiocyanate (FITC)-labeled catalase (FIG. 1D).

To determine the oxygen-release kinetics, an oxygen-sensitive luminophore, Ru(Ph2phen3)Cl2, whose fluorescence intensity is linearly proportional to the oxygen content, was used. The ORMs were able to continuously release oxygen during the 2-week experimental period (FIG. 1E). The oxygen level reached above 5% after 2 days of release, and it was maintained above 10% from days 3 to 14. After the 2-week study period, the release medium contained less than 10 μM H2O2, as measured by a quantitative peroxide assay kit. This concentration will not cause cell apoptosis.

### Continuous Oxygenation of Skin Cells by Released Oxygen Promoted Skin Cell Survival, Migration and Paracrine Effects, and Endothelial Lumen Formation Under Hypoxia In Vitro

In diabetic wounds, hypoxia is a major factor compromising cell survival and migration, leading to slow wound healing. To evaluate whether oxygen released from ORMs was able to improve cell survival under hypoxia, human keratinocytes (HaCaT cells), human dermal fibroblasts (HDFs), and human arterial endothelial cells (HAECs) were incubated with the ORMs under 1 oxygen. All three cell types exhibited a significantly higher double-stranded DNA (dsDNA; characteristic of live cells) content than the corresponding control groups without ORMs (P<0.001 for HaCaT cells and HAEC and P<0.01 for HDF; FIG. 1F-H). These results demonstrate that the released oxygen effectively increased skin cell survival under hypoxia. One of the concerns of oxygen treatment is the overproduction of ROS. To characterize ROS expression in the three cell types, an ROS-sensitive dye, CM-H2DCFDA, was used to stain the cells cultured under normoxia, hypoxia (1% oxygen), and hypoxia with the addition of ORMs (hypoxia/ORM group). The released oxygen in the hypoxia/ORM group substantially increased the ROS level in the three cell types compared to the hypoxia group (FIGS. 1I-K, 10, and 12). However, the ROS level was similar to the normoxia group (P>0.05 for all cell types). These results, together with dsDNA results, show that the released oxygen did not overproduce ROS and induce cell apoptosis. To determine whether the released oxygen could increase the migration of keratinocytes and dermal fibroblasts, a scratch assay was performed. After 48 hours of incubation under 1% oxygen, the migration rates of HaCaT cells and HDFs were significantly higher in the ORM groups than in the control groups without ORMs (P<0.001; FIGS. 1L, M, O, and P). These results demonstrate that the released oxygen promoted skin cell migration under hypoxia.

The effect of released oxygen on skin cell expression of angiogenic growth factors under hypoxic conditions was further elucidated. For HaCaT cells, treatment with ORMs significantly increased the expression of vascular endothelial growth factor-A (VEGFA; P<0.001) (FIG. 1N). For HDFs, the released oxygen significantly augmented the expression of platelet-derived growth factor-B (PDGFB; P<0.001) and FGF2 (P<0.001) (FIG. 1Q). These growth factors play critical roles in reepithelialization and angiogenesis during cutaneous wound healing. It remains to be investigated whether the up-regulation of these growth factors is associated with a signaling cascade, which can be activated by the released oxygen.

In chronic wounds, the hypoxic environment impairs angiogenesis, resulting in delayed wound healing. To evaluate the potential of released oxygen in promoting angiogenesis under hypoxia, an in vitro endothelial tube formation assay was conducted (FIG. 1R). The ORM was injected into three-dimensional (3D) collagen constructs seeded with HAECs. Following 16 hours of culture under 1% oxygen, the HAECs assembled with a significantly greater number of lumens, exhibiting an ˜2.5-fold increase in lumen density than the control group (P<0.01, FIGS. 1S and T).

### Continuous Oxygenation by Released Oxygen Elevated Intracellular Oxygen Content and Adenosine Triphosphate Content and Activated Extracellular Signal-Regulated Kinase 1/2 and Heme Oxygenase 1 Signaling

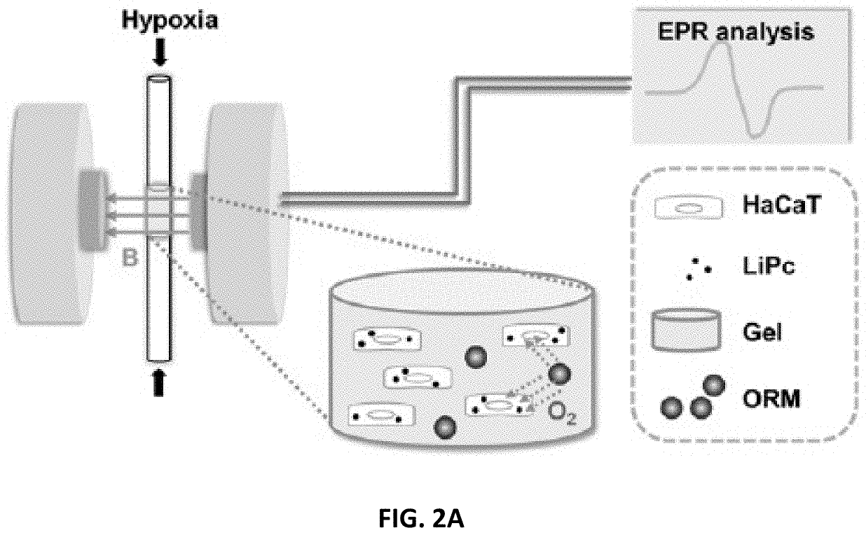

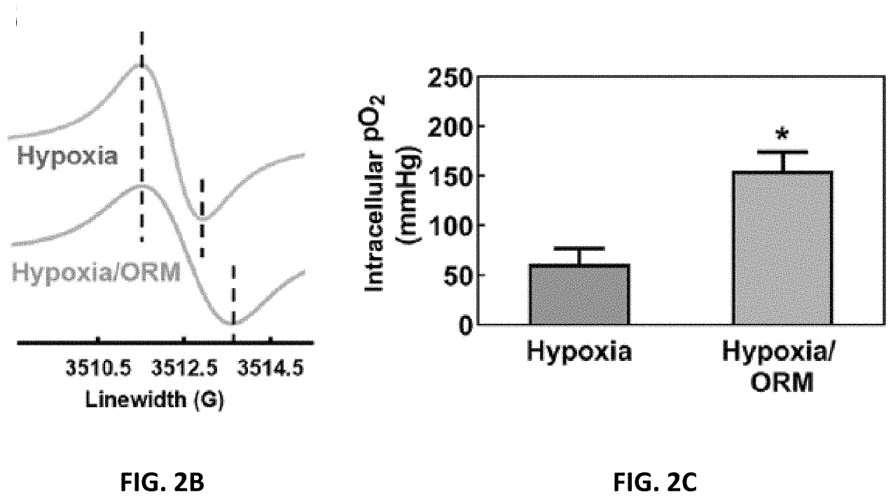

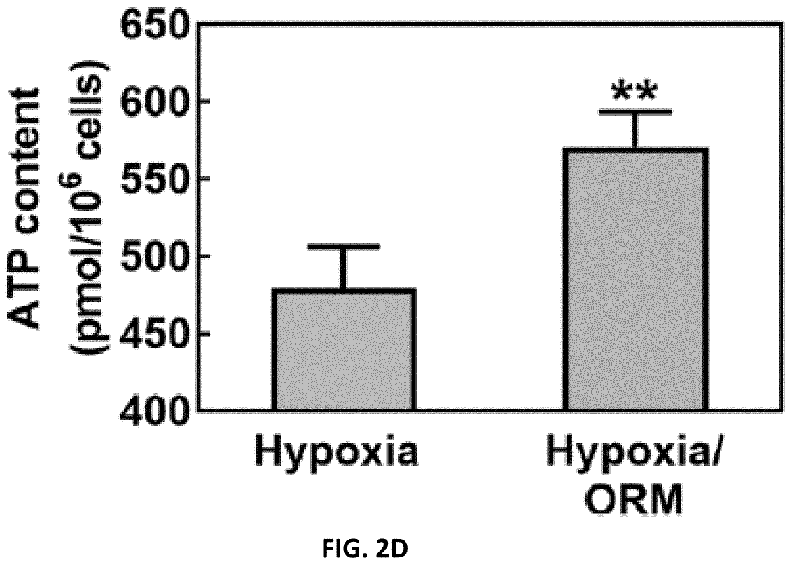

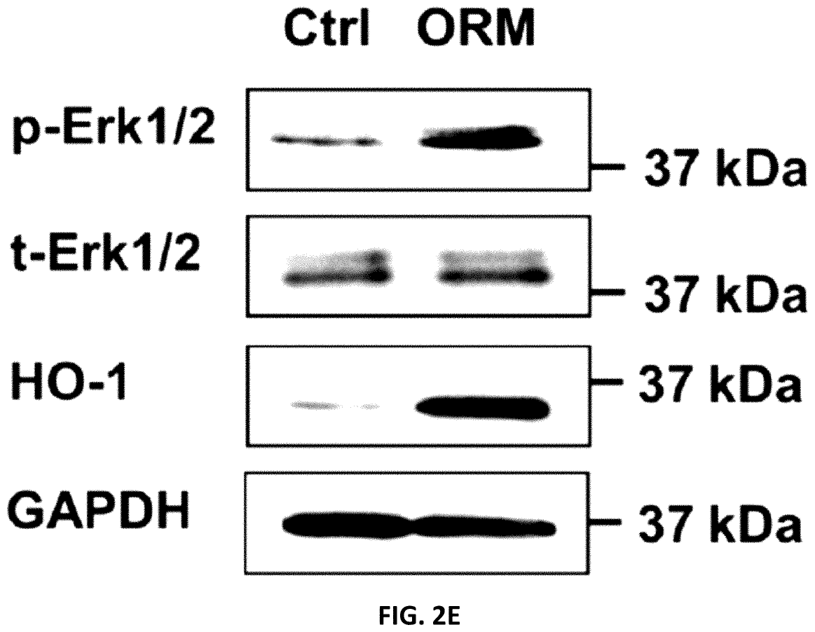

To explore how continuous oxygenation by released oxygen increased skin cell survival and migration, electron paramagnetic resonance (EPR) was used to measure the intracellular oxygen content in HaCaT cells (FIG. 2A). After 24 hours of treatment with ORMs under 1% oxygen, the intracellular oxygen content was 2.5 times that of cells without ORM treatment (FIGS. 2B and C). To determine whether the elevated intracellular oxygen content promoted cellular energy production, the intracellular adenosine triphosphate (ATP) level in HaCaT cells was measured. The ATP level in the group treated with ORMs was significantly increased over that in the group without ORM treatment (P<0.01; FIG. 2D), demonstrating that increased cell survival and migration by continuous oxygenation is associated with increased intracellular oxygen content and energy generation.

Oxygen-mediated wound healing is associated with various signaling molecules or pathways, such as mitogen-activated protein kinase (MAPK), transforming growth factor-β1, heme oxygenase 1 (HO-1), and heat shock proteins. Many researchers have confirmed that HBOT activates the MAPK pathway, which modulates cellular responses such as proliferation and migration. Thus, whether the ORMs can increase MAPK activity to promote the survival and migration of skin cells was investigated. Western blot analysis was performed using HDF to determine whether the extracellular signal-regulated kinase (Erk1/2) axis, one of the major cascades of the MAPK pathway, was activated in response to the released oxygen. The results demonstrated that Erk1/2 phosphorylation was increased after the cells were treated with ORMs under 1% oxygen (FIG. 2E). In addition, whether HO-1, a stress-inducible and cytoprotective protein, was up-regulated after treatment with released oxygen under hypoxia was investigated. In the ORM group, the HO-1 expression was more pronounced than in the control group (FIG. 2E). Together, the above results reveal that continuous oxygenation by released oxygen activated the Erk1/2 and HO-1 pathway, leading to enhanced cell survival and migration.

### The Injectable, Thermosensitive, and ROS-Scavenging Hydrogel Delivered ORMs and Protected Skin Cells Under Oxidative Stress

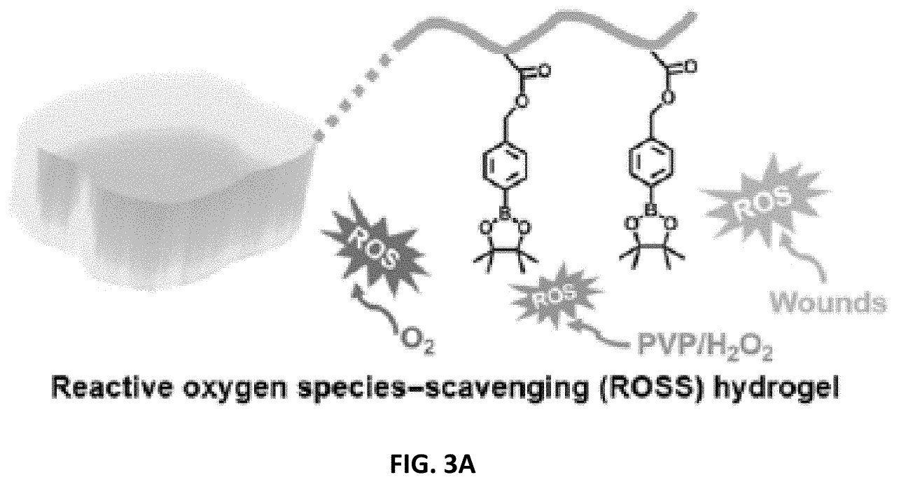

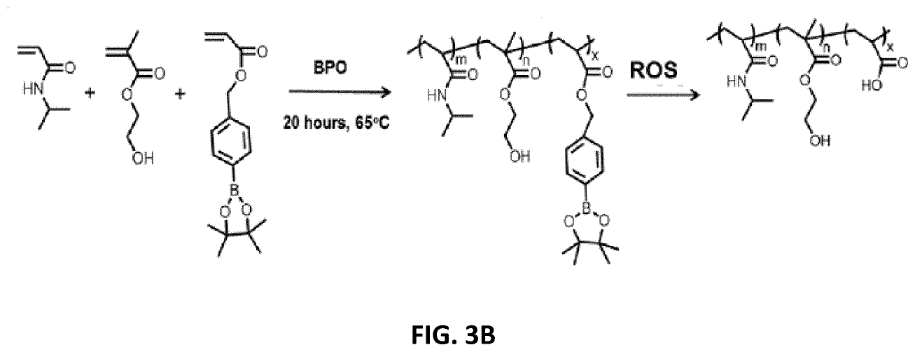

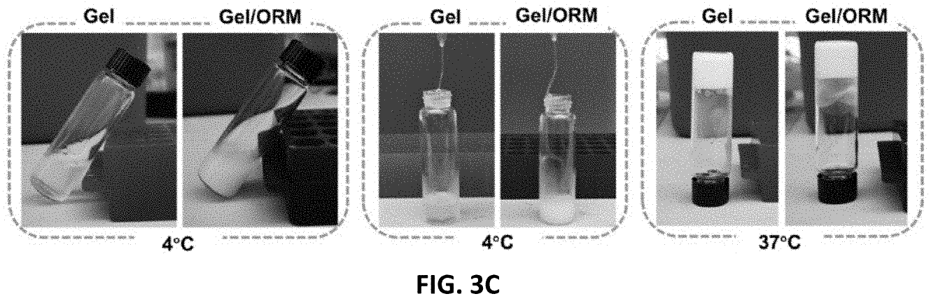

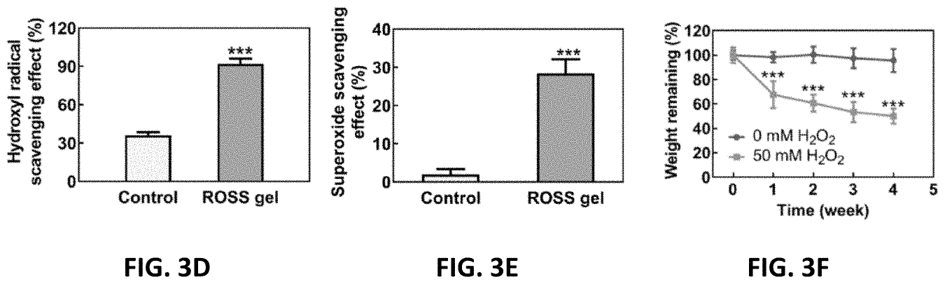

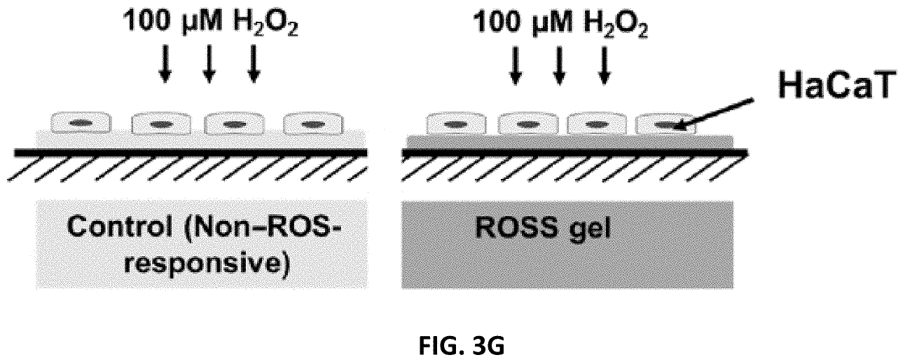

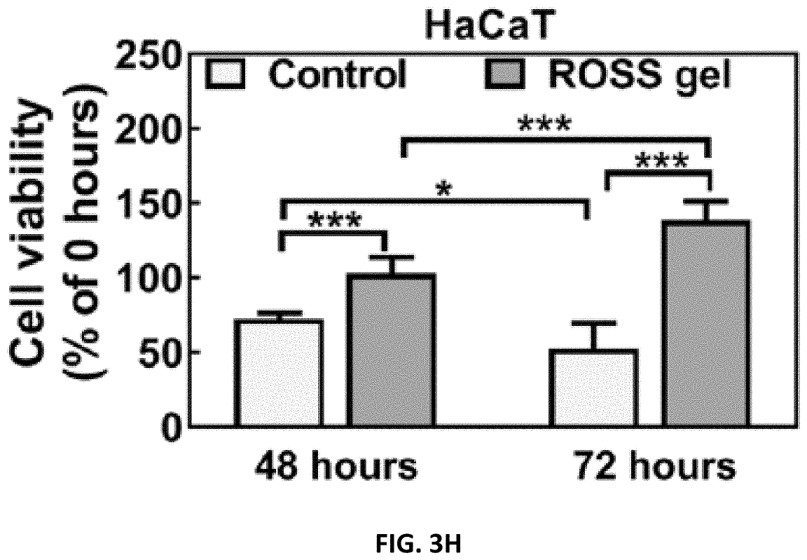

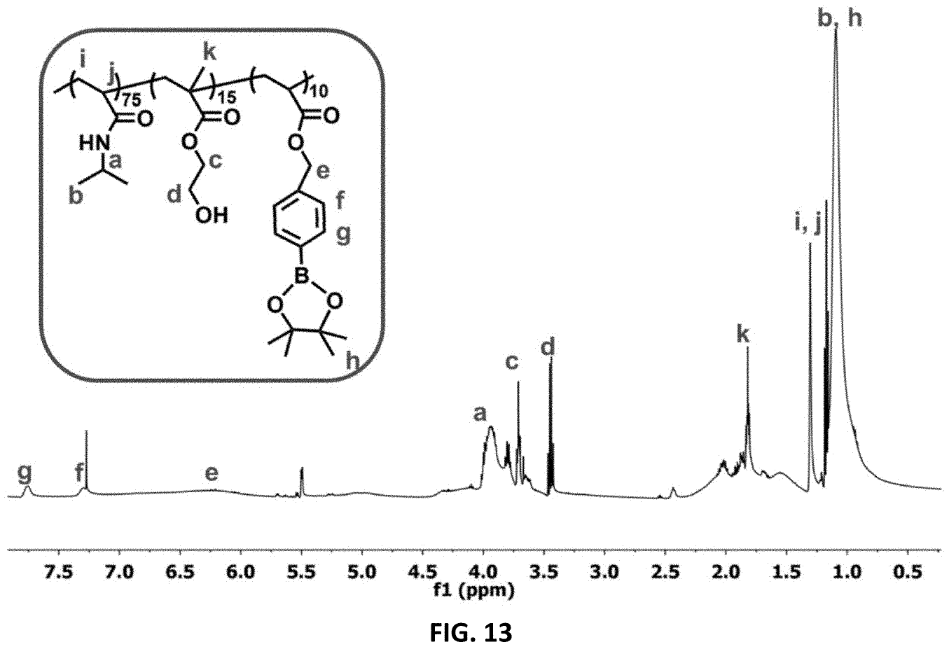

To deliver the ORMs to diabetic wounds and largely retain them in the tissue, an injectable and thermosensitive hydrogel with a fast gelation rate was synthesized. The hydrogel was also designed to capture up-regulated ROS in diabetic wounds to protect skin cells from ROS-induced apoptosis. This hydrogel may also eliminate ROS generated by excessive oxygen release. The hydrogel was synthesized by copolymerizing NIPAAm, HEMA, and 4-(acryloyloxymethyl)-phenylboronic acid pinacol ester (FIGS. 3A, 3B, and 13). The hydrogel solution [6 weight % (wt %)] had a gelation temperature of 17° C., and it was injectable at 4° C. After being transferred into a 37° C. water bath, the hydrogel solution solidified within 6 s (FIG. 3C). The mixture of hydrogel solution and ORMs (40 mg/ml) remained injectable at 4° C. and gelled quickly (6 s) at 37° C. (FIG. 3C).

The ROS-scavenging capability of the hydrogel was evaluated in terms of its consumption of hydroxyl radical (HO•) and superoxide (O2•-) and by the hydrogel weight loss induced by H₂O₂. After incubation with HO for 1 day, the hydrogel scavenged markedly more HO• than the non-ROS-responsive control hydrogel (P<0.001; FIG. 3D). Similarly, the hydrogel eliminated eightfold more O2•- than the control hydrogel after incubation for 1 day (P<0.001; FIG. 3E). The hydrogel gradually lost weight in H₂O₂ solution during the 4-week experimental period (FIG. 3F). In contrast, the hydrogel did not show substantial weight loss in Dulbecco's phosphate-buffered saline (DPBS) without H₂O₂.

To evaluate the efficacy of the hydrogel in protecting skin cell survival under pathological oxidative stress, HaCaT cells were seeded on the hydrogel surface in the presence of 100 μM H₂O₂, and the cell viability was quantified (FIG. 3G). The non-ROS-responsive hydrogel was used as a control. It was found that HaCaT cells seeded on the hydrogel did not undergo substantial apoptosis in 100 μM H₂O₂ at 48 hours and even proliferated at 72 hours. However, HaCaT cells on the non-ROS-responsive hydrogel had significantly lower viability than the ROS-responsive hydrogel (P<0.001 at 48 and 72 hours), with only ˜50% viability at 72 hours (FIG. 3H). These results demonstrate that the ROS-responsive hydrogel was able to protect keratinocytes from apoptosis caused by pathological oxidative stress.

### Diabetic Wound Closure was Accelerated by Continuous Oxygenation and ROS Scavenging

To determine whether continuous oxygenation and ROS scavenging can accelerate diabetic wound healing, the hydrogel with ORMs (Gel/ORM group) was administered onto full-thickness excisional wounds on diabetic mice (FIG. 4A). Wounds without treatment (No treatment group) and wounds treated with hydrogel alone (Gel group) were used as controls. The wounds treated with Gel/ORM had a greater closure rate than the No treatment and Gel groups (FIG. 4B). By day 16, the wound size in the Gel/ORM group was reduced to 10.7%, significantly smaller than that in the Gel (30.4%) and No treatment groups (52.2%) (P<0.01; FIG. 4C). Notably, the wound size in the Gel group was substantially smaller than in the No treatment group, demonstrating that the Gel alone promoted wound closure.

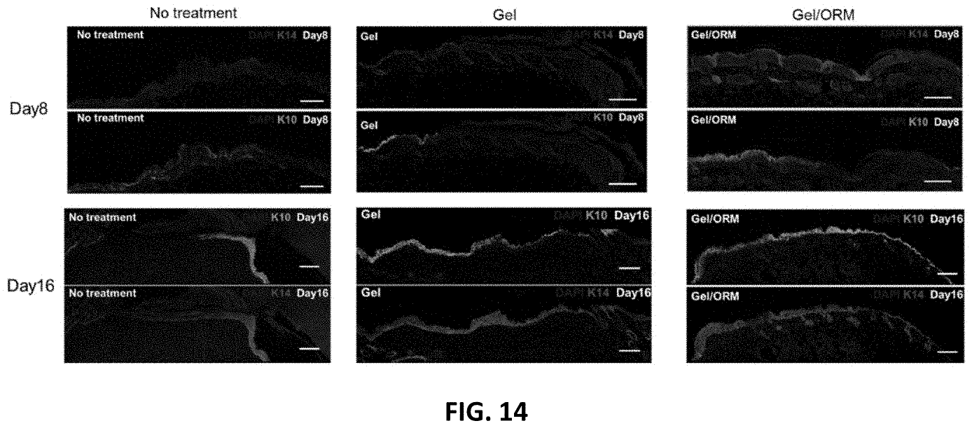

Reepithelialization is crucial in wound healing. To evaluate the rate of reepithelialization, cytokeratin 14 (K14) and cytokeratin 10 (K10) were used, two markers for basal keratinocytes and spinous keratinocytes, respectively, to stain tissue sections harvested on days 8 and 16. On day 8, in the Gel/ORM group, the wound gap was nearly enclosed by a complete layer of basal keratinocytes (K14+), while in the No treatment and Gel groups, keratinocyte migration was much slower (FIGS. 4D and 14). In addition, compared to the No treatment and Gel groups, the Gel/ORM group formed a more mature spinous layer composed of K10+ keratinocytes. On day 16, the migration of basal keratinocytes was complete, and proliferation and differentiation were ongoing in the No treatment and Gel groups. In contrast, reepithelialization was fully complete in the Gel/ORM group, as evidenced by a clear stratified epithelium (FIGS. 4D and 14). On both day 8 and day 16, K14 staining of the hair follicles in the basal layer of the epidermis (FIG. 4E) revealed a significantly increased hair follicle density in the Gel/ORM group than in the No treatment and Gel groups (P<0.01, FIG. 4F). These results demonstrate that the continuous oxygenation by released oxygen, together with ROS scavenging, effectively accelerated keratinocyte migration and hair follicle formation, consequently promoting reepithelialization of the diabetic wounds. Epidermal thickness, another indicator of wound healing, increases during the initial inflammatory and proliferative stages and then decreases during the remodeling stage. On day 8, the wounds treated with Gel/ORM exhibited the thickest epidermis. By day 16, it had become the thinnest (P<0.001; FIGS. 4G and H), suggesting that the continuous oxygenation and ROS scavenging facilitated the wound's healing progressions from the inflammatory and proliferative stages to the remodeling stage.

To determine whether the accelerated wound closure was associated with scar formation, picrosirius red staining for collagens in the wounds was performed. On day 16, the Gel/ORM group had a significantly lower total collagen content than the No treatment group (P<0.05; FIGS. 4I and J). In addition, a significantly higher collagen I/III ratio was found for the Gel/ORM group than the No treatment and Gel groups (P<0.05; FIG. 4K). Compared to the control groups, this collagen I/III ratio was closer to that of uninjured skin. The reduced collagen deposition and higher collagen I/III ratio demonstrate that the enhanced wound healing did not induce scar formation.

### Continuous Oxygenation and ROS Scavenging in Diabetic Wounds Promoted Cell Proliferation and Metabolism

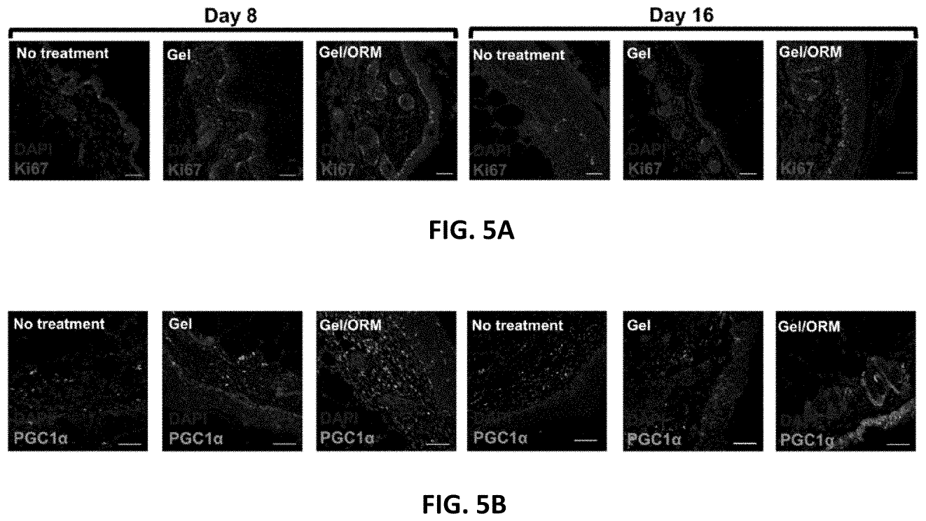

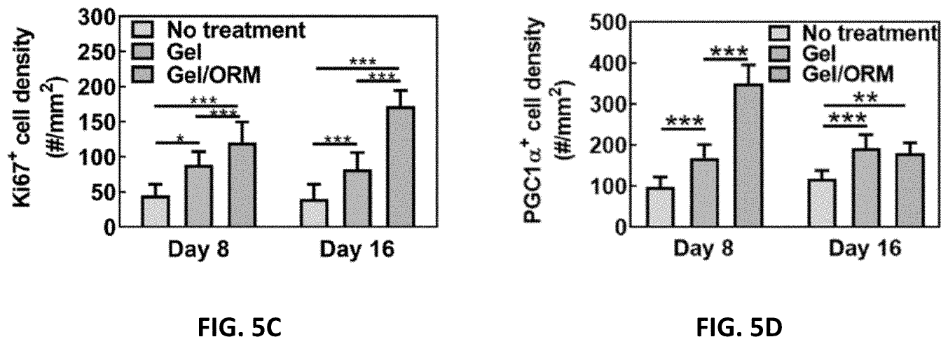

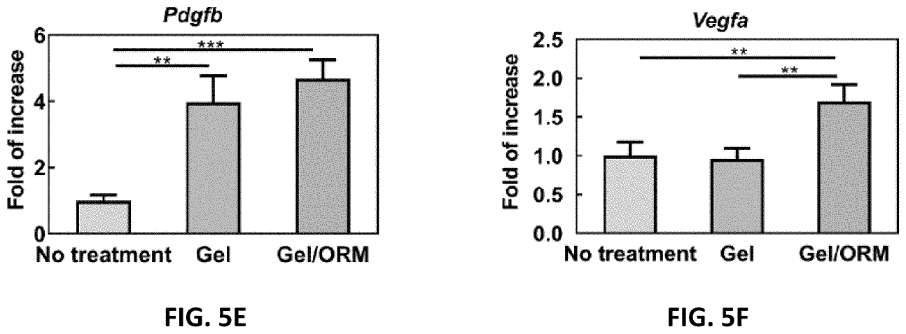

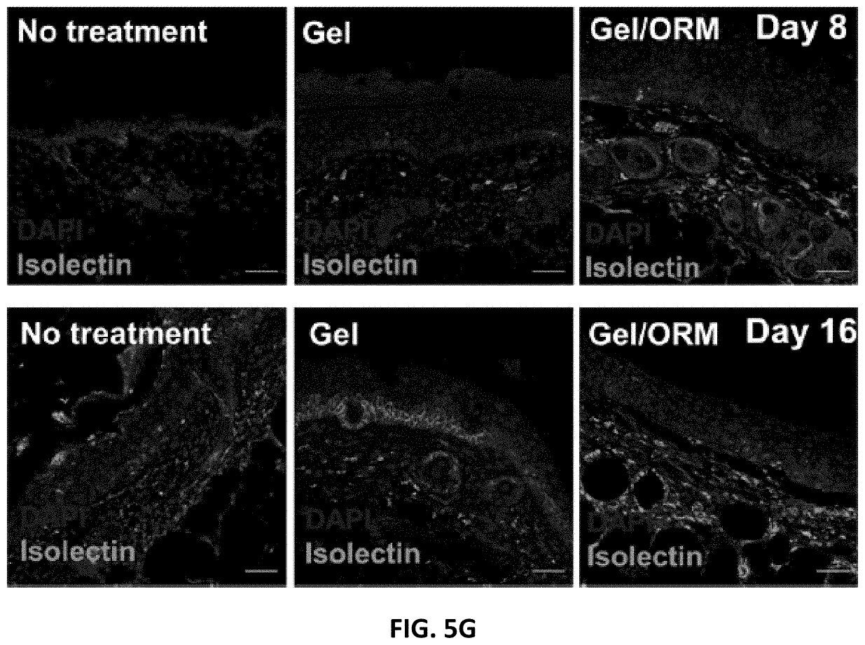

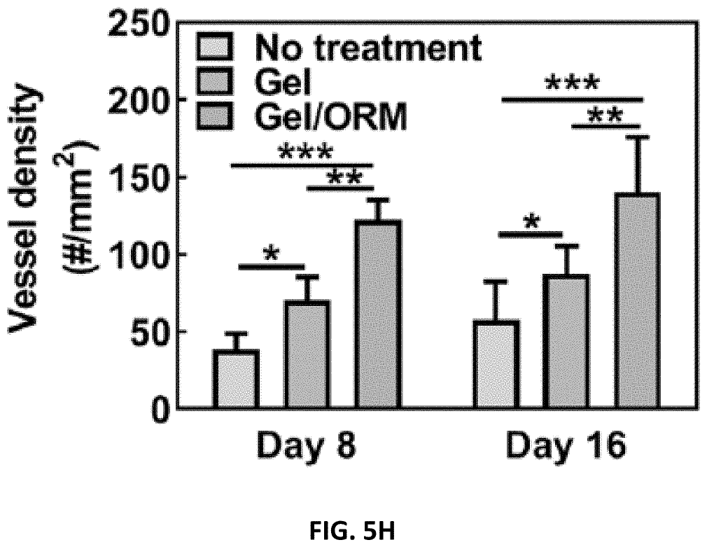

To understand the role of continuous oxygenation and ROS scavenging in diabetic wound healing at the cellular level, cell proliferation and metabolism in the wounds were examined. Compared to the No treatment group on both day 8 and day 16, the ROS-scavenging activity of the hydrogel alone had significantly increased the density of Ki67+ proliferating cells. On day 16, the density of proliferating cells treated with Gel/ORM had increased further (FIGS. 5A and C).

Continuous oxygenation and ROS scavenging also increased the skin cell metabolic rate, as judged by the peroxisome proliferator-activated receptor gamma coactivator 1α (PGC1α) positive cell density (FIGS. 5B and D). Compared with the No treatment group, the Gel group exhibited a significantly higher density of PGC1α+ cells. The release of oxygen in the Gel/ORM group further increased the PGC1α+ cell density at both time points.

### Continuous Oxygenation and ROS Scavenging in Diabetic Wounds Stimulated Angiogenic Growth Factor Expression and Angiogenesis

To determine whether continuous oxygenation and ROS scavenging affect the expression of angiogenic growth factors in diabetic wounds, real-time reverse transcription polymerase chain reaction (RT-PCR) was performed using wound tissues extracted 8 days after surgical wounding (FIGS. 5E and F). Among different angiogenic growth factors, Pdgfb expression was significantly increased in both the Gel and Gel/ORM groups (P<0.01; FIG. 5E), with the Gel/ORM group showing substantially greater expression than the Gel group (P>0.05). Notably, the Gel/ORM group exhibited significantly higher Vegfa expression than the Gel and No treatment groups (P<0.01; FIG. 5F). To evaluate whether continuous oxygenation and ROS scavenging stimulated angiogenesis in the diabetic wounds, the capillary densities in the Gel/ORM, Gel, and No treatment groups at middle stage (day 8) and late stage (day 16) of the wound healing were quantified. At both stages, the Gel group had a significantly higher density of capillaries than the No treatment group, demonstrating that ROS scavenging promoted diabetic wound angiogenesis (FIGS. 5G and H). The simultaneous ROS scavenging and continuous oxygenation in the Gel/ORM group further significantly stimulated angiogenesis (FIGS. 5G and H).

### Continuous Oxygenation and ROS Scavenging in Diabetic Wounds Alleviated Oxidative Stress, Inflammation, and Proinflammatory Cytokine Expression

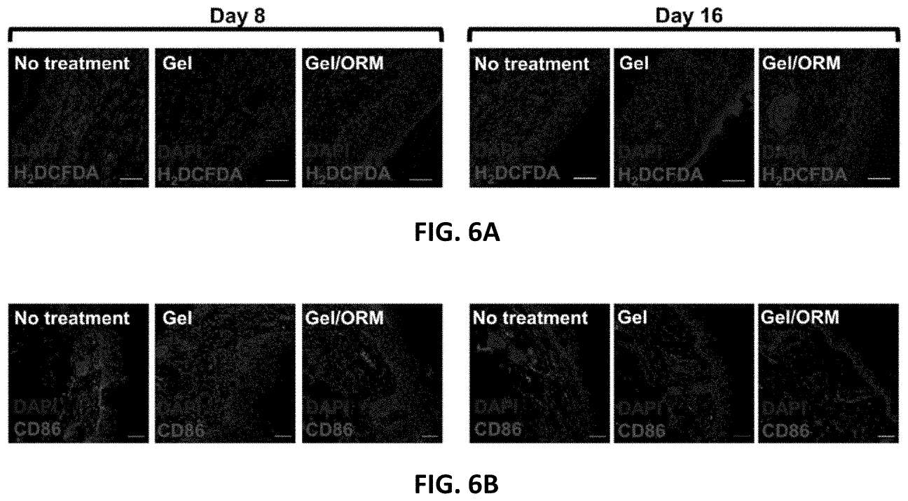

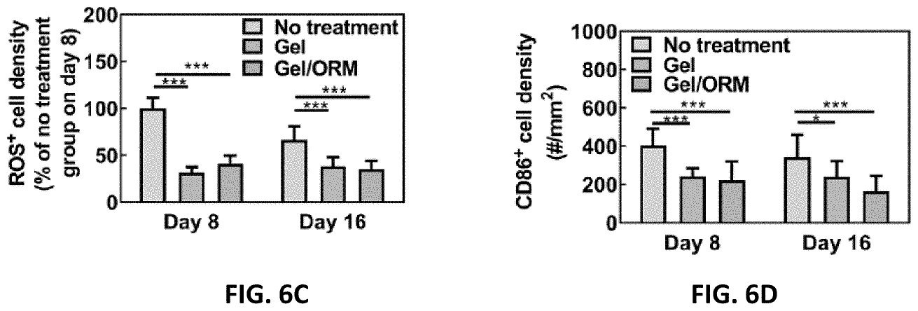

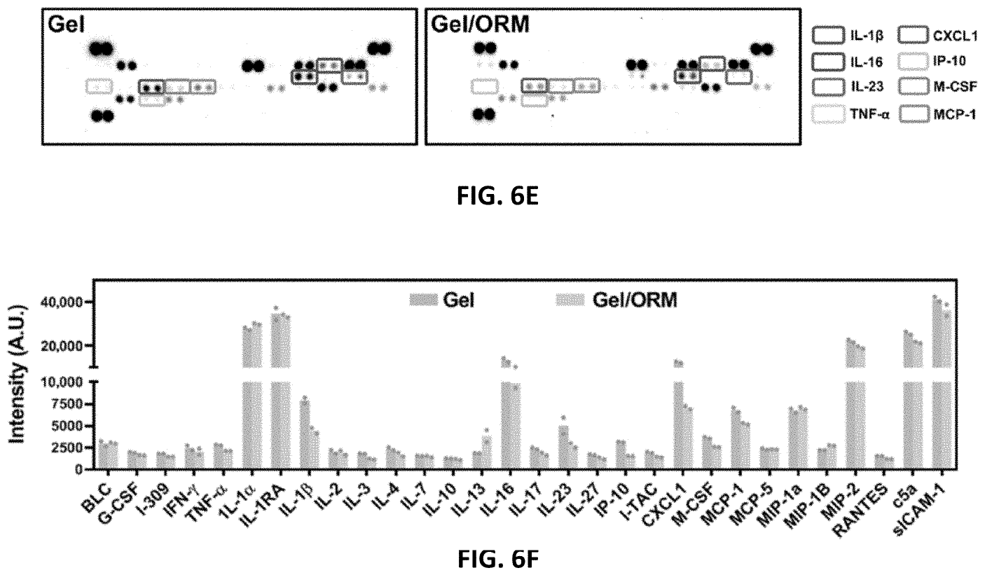

The ROS-scavenging hydrogel decreased the ROS content in diabetic wounds (FIGS. 6A and C). On both day 8 and day 16, the ROS+ cell density was markedly lower in the Gel group than in the No treatment group (P<0.001). Although the continuous oxygenation of the diabetic wounds may have led to the formation of ROS, the hydrogel was able to capture it. On days 8 and 16, the Gel and Gel/ORM groups showed similar densities of ROS+ cells (P>0.05, FIGS. 6A and C), demonstrating that the hydrogel can efficiently scavenge ROS even if continuous oxygenation induces ROS formation. To elucidate the impact of continuous oxygenation and ROS scavenging on the inflammatory response in diabetic wounds, we evaluated inflammatory cell density in the tissue by CD86 staining. The CD86+ inflammatory cell density in the Gel and Gel/ORM groups was significantly lower than in the No treatment group (with P<0.001 on day 8 and P<0.05 on day 16; FIGS. 6B and D). Notably, the Gel and Gel/ORM groups had similar inflammatory cell densities. These results demonstrate that ROS scavenging by the hydrogel decreased diabetic wound inflammation. To further reveal the role of released oxygen in tissue inflammation, we assessed proinflammatory cytokine expressions in diabetic wounds (FIGS. 6E and F). Compared with the Gel group, the Gel/ORM group exhibited reduced expression of various proinflammatory cytokines, such as interleukin-1β (IL-1β), tumor necrosis factor-α (TNF-α), interferon-γ (IFN-γ), and chemokine ligand 1 (CXCL1). These results suggest that continuous oxygenation has the potential to decrease inflammation during diabetic wound healing.

### Continuous Oxygenation in Diabetic Wounds Up-Regulated Phosphorylated Erk1/2 and HO-1 Expressions

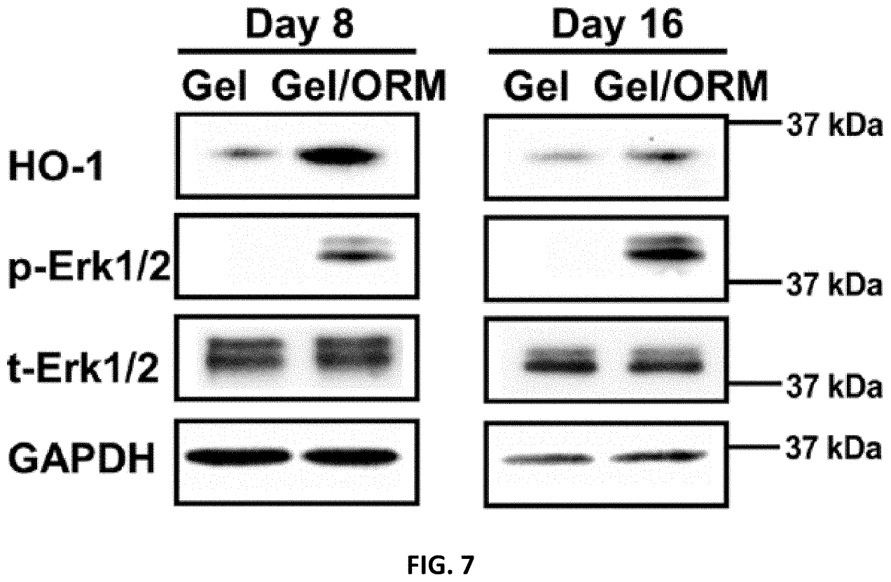

After showing that the oxygenation of keratinocytes by released oxygen up-regulated phosphorylated Erk1/2 (p-Erk1/2) and HO-1 expressions in vitro, the role of continuous oxygenation in up-regulating these expressions in diabetic wounds was next evaluated. Consistent with the in vitro findings, the p-Erk1/2 and HO-1 expressions were substantially up-regulated in the Gel/ORM group on days 8 and 16 (FIG. 7).

### Discussion:

Chronic hypoxia is a main characteristic of diabetic wounds and a severe impediment to the healing process. Consequently, sustained oxygenation of skin cells to mitigate chronic hypoxia represents an approach to accelerating diabetic wound healing. Oxygen therapy is advantageous over drug therapy because it raises fewer toxicity concerns. However, current oxygen therapy approaches cannot provide sufficient oxygen to metabolic-demanding skin cells long enough to promote diabetic wound healing. In this work, an oxygen-release system that continuously oxygenates diabetic wounds was developed. It is composed of ORMs and their carrier, an injectable, thermosensitive, fast-gelling, and ROS-scavenging hydrogel. The ORMs have a core-shell structure, with PVP/H₂O₂ complex as the core and a bioeliminable polymer as the shell. The high-molecular weight PVP/H₂O₂ complex reduces H₂O₂ diffusivity, allowing for sustained release of H₂O₂ during the hydrolysis of the shell polymer. The ORM surface is conjugated with catalase to timely convert H₂O₂ in the released PVP/H₂O₂ into molecular oxygen (FIG. 1A). Therefore, the ORMs can directly release oxygen (FIG. 1E), whereas most other oxygen-release systems directly release H₂O₂ instead of oxygen. It was demonstrated that the microspheres could release oxygen for at least 2 weeks (FIG. 1E), longer than most other oxygen-release systems. The injectable, thermosensitive, and fast-gelling hydrogel used as the ORM carrier largely retained the ORMs in the diabetic wounds after delivery. The ROS-scavenging hydrogel both eliminates the H₂O₂ in the released PVP/H₂O₂, even if it is not completely converted by catalase, and captures the up-regulated H₂O₂ in diabetic wounds to decrease oxidative stress and accelerate wound healing. To the best of available knowledge, no current oxygen-release system has been designed to release molecular oxygen and scavenge ROS simultaneously.

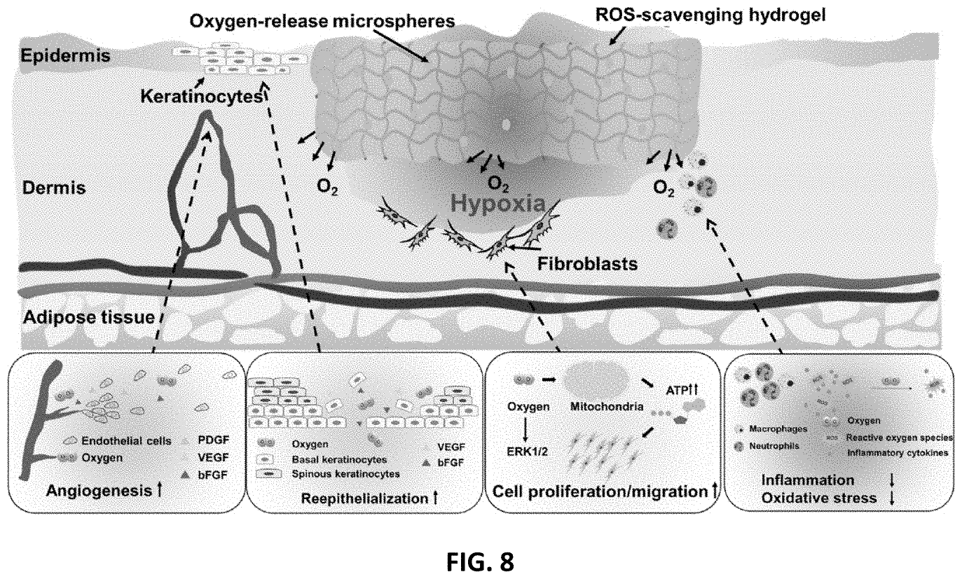

The efficacy and mechanism of action of the oxygen-release system in healing excisional wounds were evaluated in db/db mice (FIG. 4A). This model exhibits a significant delay in wound closure and impaired wound bed vascularization compared with other well-accepted murine diabetes models, such as streptozocin-induced C57BL/6J and Akita mice. The results showed that the oxygen-release system substantially promoted wound closure (FIG. 4C). More specifically, it was sought to demonstrate that the accelerated wound healing was caused by the sustained oxygenation and ROS scavenging in diabetic wounds that augmented cell survival, accelerated cell migration, stimulated angiogenesis, and reduced oxidative stress and inflammation (FIG. 8).

First, the effect of oxygen released from ORMs on skin cell survival in a hypoxic environment was investigated. In vitro studies were conducted under 1 O₂ and high-glucose conditions to mimic the in vivo microenvironment in diabetic wounds. The survival of keratinocytes, fibroblasts, and endothelial cells under hypoxia was significantly increased with the supplement of oxygen released from the ORMs (FIG. 1F to H). These three cell types are responsible for epithelialization, wound contraction, and angiogenesis. In diabetic wounds, treatment with the oxygen-release system significantly increased the density of proliferating cells (FIGS. 5A and C). The augmented cell survival can be attributed to the elevated cellular oxygen content (FIG. 2C). Cellular oxygen is essential for mitochondrial metabolism, and the low oxygen level in diabetic wounds impairs this process. The release of oxygen significantly increased the mitochondrial metabolism of the cells in the wounds on day 8, as evidenced by greater PGC1α+ cell density (FIGS. 5B and D). Mechanistically, it was found that the enhanced skin cell survival with released oxygen is associated with the up-regulation of p-Erk1/2 in vitro and in vivo (FIGS. 2E and 7).

Next, it was demonstrated that continuous oxygenation of skin cells promoted the migration and paracrine effects of keratinocytes and fibroblasts under hypoxia (FIG. 1L-Q). The migration of these cells is essential for the regeneration of the epidermis and dermis. The enhanced paracrine effects, in terms of up-regulation of growth factors such as VEGFA, FGF2, and PDGFB, facilitate the regeneration. Specifically, VEGFA and FGF2 have been found to increase keratinocyte migration and proliferation. The up-regulated expression of VEGFA, FGF2, and PDGFB in skin cells in response to released oxygen may be attributed to increased p-Erk1/2 expression (FIG. 2E). Previous studies have demonstrated that activation of the Erk1/2 pathway was able to up-regulate cell angiogenic growth factor expression. The enhanced keratinocyte survival, migration, and paracrine effects of continuous oxygenation led to a significant increase in the wound closure rate (FIG. 4C) and the formation of stratified epithelium (FIG. 4D).

Angiogenesis in diabetic wounds is essential for the regeneration of the dermis. Although acute hypoxia induces angiogenesis mediated by hypoxia-inducible transcription factors, chronic oxygen deprivation cannot sustain this process, thereby impairing the healing process. It was found that the released oxygen increased endothelial cell survival (FIG. 1H) and tube formation (FIG. 1R-T) in vitro. In diabetic wounds, continuous oxygenation significantly stimulated capillary formation (FIGS. 5G and H). The quicker angiogenesis may also be attributed to the enhanced paracrine effects because the expressions of angiogenic growth factors Pdgfb and Vegfa were substantially up-regulated (FIGS. 5E and F).

Furthermore, it was shown that the developed oxygen delivery system alleviated oxidative stress in diabetic wounds (FIGS. 6A and C). The decreased oxidative stress was due to the ROS-scavenging hydrogel since the ROS+ cell densities in the Gel and Gel/ORM groups were similar. This result minimizes the potential concern that oxygen release may lead to ROS overproduction. The decreased oxidative stress may also be associated with increased HO-1 expression under hypoxic conditions (FIGS. 2E and 7). HO-1 has antioxidant effects and has been reported to promote diabetic wound healing.

Last, it was shown that the oxygen-release system reduced inflammation in diabetic wounds (FIGS. 6B and D). Chronic wounds are characterized by a high concentration of proinflammatory cells, the major contributor of proinflammatory cytokines, such as TNF-α, IL-1β, and CXCL1. In addition, hyperglycemia exacerbates inflammatory stress via the production of proinflammatory cytokines such as TNF-α. The results show that ROS-scavenging hydrogel greatly decreased the number of inflammatory cells (FIGS. 6B and D) and the released oxygen further reduced the expression of major proinflammatory cytokines (FIGS. 6E and F). Future studies will focus on delineating signaling pathways as the underlying mechanism in suppressing inflammation by sustained oxygen release in diabetic wounds.

Overall, it was demonstrated in this report that a sustained oxygen-release system that simultaneously scavenges ROS substantially accelerated diabetic wound closure. The sustained release of oxygen had multiple effects: It promoted skin cell survival, migration, and paracrine effects; stimulated endothelial tube formation and angiogenesis; and decreased tissue inflammation. Compared with the results of previous studies using growth factors or exogenous cells to treat impaired wound healing in the same animal model, this oxygen-release system exhibited faster or similar wound closure rates. Thus, it represents an effective therapeutic approach for the accelerated healing of chronic diabetic wounds without using drugs. Beyond wound healing, the developed oxygen-release system may be used to treat other ischemic diseases, such as peripheral artery disease and coronary heart disease. Given the limitations of rodent models in illustrating more complex human pathophysiology, the oxygen-release system will be further tested in large animals and the oxygen-release kinetics and ROS-scavenging capability will be optimized accordingly.

### Materials and Methods:

### Materials

All chemicals were purchased from MilliporeSigma unless otherwise stated. NIPAAm (TCI) was recrystallized in hexane before use. HEMA (Alfa Aesar) was used before passing through a column filled with inhibitor removers. Benzoyl peroxide (BPO; Thermo Fisher Scientific), acryloyl chloride, 4-(hydroxymethyl)-phenylboronic acid pinacol ester, triethylamine (Fisher Scientific), PVP (40 kDa; Fisher Scientific), hydrogen peroxide solution (30%), and bovine liver catalase (2000 to 5000 U/mg) were used as received.

### Synthesis and Characterization of ROS-Scavenging Hydrogel

4-(acryloxymethyl)-phenylboronic acid pinacol ester was synthesized by the reaction between acryloyl chloride and 4-(hydroxymethyl)-phenylboronic acid pinacol ester. The ROS-scavenging hydrogel was synthesized by free radical polymerization of NIPAAm, HEMA, and 4-(acryloxymethyl)-phenylboronic acid pinacol ester using BPO as initiator and 1,4-dioxane as solvent. The reaction was conducted at 70° C. overnight with the protection of nitrogen. The polymer solution was precipitated in hexane. The polymer was purified twice by dissolving in tetrahydrofuran and precipitating in ethyl ether. The polymer was dissolved in DPBS at 4° C. to make a 6 wt % solution. The injectability of the 4° C. solution with or without ORMs (40 mg/ml) was tested by a 26-gauge needle.

Hydrogel H₂O₂ responsiveness was characterized by its weight loss after being incubated in an H₂O₂ solution for 4 weeks. Briefly, the hydrogel solution was solidified in 1.5 ml microcentrifuge tubes at 37° C. After the supernatant was discarded, 0.2 ml, 37° C. DPBS with or without 50 mM H₂O₂ was added. The samples were collected at predetermined time points and freeze-dried. The weight loss was calculated. To determine the scavenging capability of the hydrogel for hydroxyl radicals and superoxide, a Fenton reaction assay and pyrogallol assay were performed, respectively. Briefly, for the Fenton reaction assay, hydrogel solution or deionized (DI) water (control group) was incubated with FeSO₄, safranin O, and H₂O₂ for 5 min, followed by heating at 55° C. for 30 min. After the samples were cooled to room temperature, the absorbance was measured at 492 nm using a microplate reader. For the pyrogallol assay, the hydrogel solution or DI water was mixed with tris-HCl. Pyrogallol solution (3 mM) was then added dropwise in the dark. The reaction was terminated by adding 8 M HCl. The absorbance was acquired at 299 nm.

Cell Survival on ROS-Scavenging Hydrogel in the Presence of H₂O₂

The ROS-scavenging hydrogel was plated on 96-well plates. HaCaT cells (AddexBio) were seeded on the hydrogel surface at a density of 50,000 per well in optimized Dulbecco's modified Eagle's medium (DMEM, AddexBio) with 5% fetal bovine serum (FBS) and 1% penicillin-streptomycin. After 24 hours of culture, the medium was discarded. A total of 200 μl of 100 μM H2O2-containing medium was added to each well. At 48 and 72 hours, the viability of HaCaT was measured by MTT assay. The non-ROS-responsive hydrogel, poly(NIPAAm-co-HEMA-co-acrylate-oligolactide), was used as a control.

### Fabrication of ORMs and Catalase Conjugation

The ORMs were fabricated by a double emulsion method. Briefly, the shell of the microspheres was synthesized by the copolymerization of NIPAAm, HEMA, NAS, and AOLA. The chemical structure of the shell polymer was confirmed by 1H-NMR (proton nuclear magnetic resonance) (FIG. 9). The synthesized polymer was dissolved in dichloromethane to form the 5 wt % oil phase. The inner water phase was prepared by dissolving 242 mg of PVP in 1 ml, 30% H₂O₂ at 4° C. overnight. The water phase was rapidly added into the oil phase and sonicated by an ultrasonic liquid processor (Cole Parmer). The primary water-in-oil emulsion was then poured into a poly(vinyl alcohol) solution to form the water-in-oil-in-water double emulsion. The mixture was stirred for 3 hours to remove dichloromethane, followed by centrifugation to collect the microspheres. The morphology and size of the microspheres were characterized by scanning electron microscopy images. To confirm the core-shell structure, FITC and rhodamine were added to the PVP/H₂O₂ solution and polymer/dichloromethane solution, respectively. The fluorescent images were acquired by a confocal microscope. To conjugate catalase onto the microsphere shell, 40 mg microspheres were mixed with 6 ml catalase solution (5 mg/ml in DI water) and stirred for 4 hours at 4° C. The mixture was then centrifuged. The microspheres were washed three times with DI water to remove unconjugated catalase. To confirm the conjugation, the catalase was prelabeled by FITC, and the fluorescent images of the microspheres were taken after the conjugation

### Oxygen-Release Kinetics

The ROS-sensitive hydrogel was dissolved in 4° C. DPBS to make a 6 wt % solution. The ORMs were then mixed with the hydrogel solution at a concentration of 40 mg/ml. The wells of a 96-well plate were covered by a polydimethylsiloxane membrane loaded with an oxygen-sensitive luminophore Ru(Ph2phen3)Cl₂ and an oxygen-insensitive dye rhodamine B. Two hundred microliters of the mixture were then added into each well (n=8 for each group). After gelation at 37° C., the supernatant was removed. A total of 200 μl of DPBS preincubated in 1% oxygen condition was added. The oxygen-release study was performed in 1% oxygen and 37° C. conditions for 2 weeks. At each time point, the fluorescent intensity for Ru(Ph2phen3)Cl₂ was measured (emission at 610 nm and excitation at 470 nm). The fluorescent intensity for rhodamine was also determined (emission at 576 nm and excitation at 543 nm) and used for normalization. Oxygen concentration was determined by the calibration curve.

### Cell Survival, Migration, and ROS Content Under Hypoxia

HaCaT cells (AddexBio) were cultured in optimized DMEM (AddexBio) with 5% FBS and 1% penicillin-streptomycin. HDF cells (Lonza) were cultured in FGM-2 BulletKit (Lonza). HAEC cells (Cell Systems) were cultured in EGM-2 BulletKit (Lonza). The medium was changed every other day.

For all in vitro studies, cells were cultured using high-glucose (450 mg/dl) and serum-free medium under 1% oxygen and 37° C. conditions. To determine cell survival, the cells were cultured in a 96-well plate using the medium with or without ORMs (40 mg/ml, n≥5 for each group). The dsDNA content was measured using a PicoGreen dsDNA assay kit (Invitrogen) after 5 days of culture for HaCaT cells and HDFs and after 7 days of culture for HAEC.

To perform the cell migration assay, the cells were first cultured in a six-well plate to reach 85 to 95% confluency (n=4 for each group). The monolayer was then scraped with a 200 μl pipette tip, washed, and supplemented with serum-free medium with or without ORMs (40 mg/ml). After 48 hours, optical images were taken using an optical microscope (Olympus IX70). The distances between two sides of the scratch were measured using ImageJ. The migration ratio was calculated as

$$migration ratio = \frac{Interval at 0 hour - interval at 48 hours}{Interval at 0 hour} \times 100 \%$$

To measure intracellular ROS content, the cells were prestained with ROS-sensitive dye CM-H2DCFDA and cultured with serum-free medium with or without ORMs (40 mg/ml). After 3 days of culture for HaCaT cells and HAEC and 5 days of culture for HDF, the cells were fixed and stained with 4′,6-diamidino-2-phenylindole (DAPI). Fluorescent images were taken using a confocal microscope. The CM-H2DCFDA positive cell density was quantified from at least 10 images for each group and then normalized to the CM-H2DCFDA positive cell density cultured under normoxia.

### Measurement of Intracellular Oxygen Content

To determine the intracellular oxygen content, HaCaT cells were incubated with lithium phthalocyanine nanoparticles for 2 hours to allow cellular uptake. The residual nanoparticles were washed with DPBS three times. After trypsinization, the cells were encapsulated in the ROS-sensitive hydrogel or mixture of hydrogel and ORMs (40 mg/ml). The samples were transferred into EPR tubes (Wilmad-LabGlass, n=3 for each group). The EPR tubes were opened on both sides and placed in a hypoxic incubator (1% oxygen, 37° C.) for 4 hours for complete gelation and gas balance. After that, the tubes were sealed and incubated for 24 hours under 1% oxygen. The EPR spectrum was recorded using an X-band EPR instrument (Bruker). The parameters used in this experiment were 0.1 mW for microwave power, 1.0 dB for attenuation, and 9.8 GHz for frequency, following our reported method. Oxygen partial pressure (pO2) was calculated from the linewidth of the spectrum and the calibration curve of linewidth versus oxygen concentration.

### Measurement of Intracellular ATP Level

To determine the intracellular ATP level, HaCaT was cultured in a six-well plate to reach 85 to 95% confluency. The culture medium was then replaced with serum-free media with or without ORMs (40 mg/ml). After 24-hour incubation under 1% O₂, the cells were lysed, and the ATP content was measured by an ATP assay kit as per the manufacturer's instruction (n=3; MilliporeSigma).

### Endothelial Tube Formation

HAECs were cultured in a 3D collagen model described previously. Briefly, the model was prepared by mixing rat tail collagen type I (Corning), FBS, DMEM, and NaOH. A total of 400 μl of the mixture was placed in a 48-well plate and incubated at 37° C. for 30 min to allow the formation of jelly-like solid collagen gel. HAECs were encapsulated in ROSS gel solution and injected into the collagen-based 3D model at a density of 10,000 cells per well. After overnight hypoxic culture, HAECs were fixed by 4% paraformaldehyde and stained with DAPI and F-actin. Fluorescent images were taken as z-stacks with a confocal microscope (Zeiss LSM 700).

Implantation of Hydrogel and ORMs into Diabetic Wounds

All animal care and experiment procedures were conducted in accordance with the National Institutes of Health guidelines. The animal protocol was approved by the Institutional Animal Care and Use Committee of Washington University in St. Louis. Eight-week-old, female db/db mice (BKS.Cg-Dock7m+/+Leprdb/J) were purchased from the Jackson laboratory. Blood glucose was measured before surgery to ensure that the glucose level was higher than 300 mg/dl. After anesthetization and removal of hair, two full-thickness, 5-mm-diameter wounds on the dorsal skin were created for each mouse using a biopsy punch. The hydrogel (6 wt % in DPBS) or Gel/ORM (6 wt % in DPBS, microspheres at 40 mg/ml) was topically administrated. Digital images of the wounds (n≥8 for each group) were taken every other day, and the wound size was determined using ImageJ. After 8 and 16 days of implantation, the mice were euthanized. The wound tissues were collected for histological and immunofluorescence analysis and RNA and protein isolation.

### Histological and Immunofluorescence Analyses

The wound tissues were fixed in 4% paraformaldehyde overnight, embedded in paraffin, and serially sectioned at 5 μm. Hematoxylin and eosin staining was performed to measure the epidermal thickness in wounded skin. Epidermal thickness was calculated in the wound-closed region. Picrosirius red staining was used to quantify the collagen deposition and collagen I/III ratio. For immunofluorescence analysis, following deparaffinization, antigen retrieval, and blocking, the sections were incubated with primary antibodies, including rabbit monoclonal anti-cytokeratin 10 (K10; Abcam, ab76318), mouse monoclonal anti-cytokeratin 14 (K14; Abcam, ab7800), rabbit monoclonal anti-CD86 (Abcam, ab234401), rat monoclonal anti-Ki67 (Invitrogen, SolA15), isolectin GS-IB4 (Invitrogen, I21411), and ROS indicator (CM-H2DCFDA; Invitrogen). The sections were then incubated with corresponding secondary antibodies. Nuclei were stained with DAPI. Quantification of the stainings was performed using ImageJ. At least six images for each group were used for the quantification.

mRNA Expression by In Vitro Cultured Cells and Diabetic Wounds

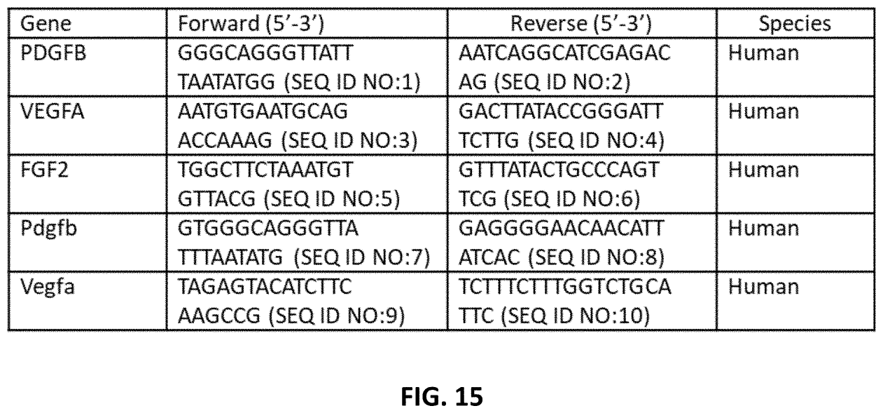

RNA was isolated from the in vitro cultured cells or wound tissues using TRIzol following the manufacturer's instructions. cDNA was synthesized using a high-capacity cDNA reverse transcription kit (Thermo Fisher Scientific). Gene expression was performed by real-time RT-PCR using SYBR Green (Invitrogen) and selected primer pairs (FIG. 15). β-Actin served as a housekeeping gene. Data analysis was performed using the ΔΔCt method.

### Protein Expression by In Vitro Cultured Cells and Diabetic Wounds

Protein lysates were collected from the in vitro cultured cells or wound tissues and separated by SDS-polyacrylamide gel electrophoresis. Low fluorescence polyvinylidene difluoride membranes (Bio-Rad) were used to transfer the proteins at 4° C. overnight, followed by blocking and incubation with primary antibodies at 4° C. The blots were washed with PBST (DPBS+0.1 Tween 20) and incubated with appropriate horseradish peroxidase-conjugated secondary antibodies. Immunoblots were detected using a detection kit from Advansta and imaged using ChemiDoc XRS+ System (Bio-Rad). The primary antibodies used were anti-glyceraldehyde-3-phosphate dehydrogenase (1:4000; Abcam, ab8245), anti-HO-1 (1:500; Abcam, ab52947), anti-p-Erk1/2 (1:500; Cell Signaling Technology, #9101), and anti-t-Erk1/2 (1:1000; Cell Signaling Technology, #4695).

For protein array assay, wounded tissue lysates were collected and tested using a Proteome profiler mouse cytokine array kit (R&D Systems) according to the manufacturer's instructions. The pixel density was quantified using Image Lab software (Bio-Rad).

### Statistical Analysis

All data were presented as means±SD. Statistical analysis was performed between groups using one-way analysis of variance (ANOVA) with Tukey's post hoc test. A value of P

### Summary:

In vitro experiments demonstrated that the ORMs released oxygen for up to 4 weeks, and no minimal residual H₂O₂ was detected throughout the release process. Additional in vitro experiments demonstrated that the ORMs enhanced the survival and proliferation of keratinocytes, endothelial cells, and fibroblasts under low oxygen and high glucose environmental conditions representative of a chronic diabetic wound.

BKS db mice were used for in vivo experiments to model phases I to III of diabetes type II. Treatment with the disclosed composition of ORMs encapsulated in ROS-sensitive hydrogel resulted in a significantly higher wound closure rate compared with a no-treatment group or a hydrogel-only group. Immunohistochemistry results demonstrated that treatment with the disclosed composition resulted in the highest density of blood vessels compared to the no-treatment group or hydrogel-only groups. No severe inflammation was observed after implantation with either hydrogel or ORMs.

The results of these experiments demonstrated that the disclosed sustained oxygen-releasing compositions facilitated wound healing in ischemic tissues such as the tissues associated with a chronic diabetic wound.

1. A composition for sustained release of oxygen to a tissue, the composition comprising at least one core-shell oxygen release microsphere (ORM), wherein the core comprises a water-soluble polymer-reactive oxygen species (ROS) complex and the shell comprises a biodegradable polymer conjugated to a ROS-scavenging enzyme.

2. The composition of claim 1, wherein the reactive oxygen species comprises hydrogen peroxide (H₂O₂).

3. The composition of claim 1, wherein the ROS-scavenging enzyme comprises catalase.

4. The composition of claim 1, wherein the water-soluble polymer comprises polyvinylpyrrolidone (PVP).

5. The composition of claim 1, wherein the biodegradable polymer comprises poly(N-isopropylacrylamide-co-2-hydroxyethyl methacrylate-co-acrylate-oligolactide-co-Nacryloxysuccinimide)[poly(NIPAAm-co-HEMA-co-AOLA-co-NAS)].

6. The composition of claim 1, further comprising a ROS-scavenging hydrogel.

7. The composition of claim 6, wherein the ROS-scavenging hydrogel comprises copolymerized NIPAAm, HEMA, and 4-(acryloyloxymethyl)-phenylboronic acid pinacol ester.

8. The composition of claim 6, wherein the hydrogel comprises at least one of a thermosensitive hydrogel, an injectable hydrogel, and any combination thereof.

9. The composition of claim 1, wherein the tissue comprises an ischemic tissue.

10. The composition of claim 9, wherein the ischemic tissue comprises a tissue associated with an ischemic condition selected from diabetes, peripheral artery disease, and coronary heart disease.

11. The composition of claim 10, wherein the ischemic tissue comprises a chronic diabetic wound bed.

12. A method for the sustained delivery of oxygen to a tissue, the method wound comprising administering a composition to the tissue, the composition comprising at least one core-shell oxygen release microsphere (ORM), wherein the core comprises a water-soluble polymer-reactive oxygen species (ROS) complex and the shell comprises a biodegradable polymer conjugated to a ROS-scavenging enzyme.

13. The method of claim 12, wherein the tissue comprises an ischemic tissue.

14. The method of claim 13, wherein the ischemic tissue comprises a tissue associated with an ischemic condition selected from diabetes, peripheral artery disease, and coronary heart disease.

15. The method of claim 14, wherein the ischemic tissue comprises a chronic diabetic wound bed.

16. A kit comprising the composition of at least one core-shell oxygen release microsphere (ORM), wherein the core comprises a water-soluble polymer-reactive oxygen species (ROS) complex and the shell comprises a biodegradable polymer conjugated to a ROS-scavenging enzyme.

17. The kit of claim 16, wherein the biodegradable polymer comprises poly(N-isopropylacrylamide-co-2-hydroxyethyl methacrylate-co-acrylate-oligolactide-co-Nacryloxysuccinimide)[poly(NIPAAm-co-HEMA-co-AOLA-co-NAS)].

18. The kit of claim 16, further comprising a ROS-scavenging hydrogel.

19. The kit of claim 18, wherein the ROS-scavenging hydrogel comprises copolymerized NIPAAm, HEMA, and 4-(acryloyloxymethyl)-phenylboronic acid pinacol ester.

20. The kit of claim 16, wherein the kit is used to treat a diabetic wound.


SEQUENCE LISTING (10 sequences)

SEQ ID NO: 1 - DNA, 21 bp, synthetic construct
  source 1..21: mol_type = other DNA
gggcagggttatttaatatgg

SEQ ID NO: 2 - DNA, 18 bp, synthetic construct
  source 1..18: mol_type = other DNA
aatcaggcatcgagacag

SEQ ID NO: 3 - DNA, 20 bp, synthetic construct
  source 1..20: mol_type = other DNA
aatgtgaatgcagaccaaag

SEQ ID NO: 4 - DNA, 21 bp, synthetic construct
  source 1..21: mol_type = other DNA
gacttataccgggatttcttg

SEQ ID NO: 5 - DNA, 20 bp, synthetic construct
  source 1..20: mol_type = other DNA
tggcttctaaatgtgttacg

SEQ ID NO: 6 - DNA, 19 bp, synthetic construct
  source 1..19: mol_type = other DNA
gtttatactgcccagttcg

SEQ ID NO: 7 - DNA, 22 bp, synthetic construct
  source 1..22: mol_type = other DNA
gtgggcagggttatttaatatg

SEQ ID NO: 8 - DNA, 20 bp, synthetic construct
  source 1..20: mol_type = other DNA
gaggggaacaacattatcac

SEQ ID NO: 9 - DNA, 20 bp, synthetic construct
  source 1..20: mol_type = other DNA
tagagtacatcttcaagccg

SEQ ID NO: 10 - DNA, 20 bp, synthetic construct
  source 1..20: mol_type = other DNA
tctttctttggtctgcattc